SEQUENCE LISTING (21 sequences)

SEQ ID NO: 1 - protein, 531 aa, homo sapiens
MAALGCARLRWALRGAGRGLCPHGARAKAAIPAALPSDKATGAPGAGPGVRRRQRSLEEIPRLGQLRFFFQLFVQGYALQLHQLQVLYKAKYGPMWMSYLGPQMHVNLASAPLLEQVMRQEGKYPVRNDMELWKEHRDQHDLTYGPFTTEGHHWYQLRQALNQRLLKPAEAALYTDAFNEVIDDFMTRLDQLRAESASGNQVSDMAQLFYYFALEAICYILFEKRIGCLQRSIPEDTVTFVRSIGLMFQNSLYATFLPKWTRPVLPFWKRYLDGWNAIFSFGKKLIDEKLEDMEAQLQAAGPDGIQVSGYLHFLLASGQLSPREAMGSLPELLMAGVDTTSNTLTWALYHLSKDPEIQEALHEEVVGVVPAGQVPQHKDFAHMPLLKAVLKETLRLYPVVPTNSRIIEKEIEVDGFLFPKNTQFVFCHYVVSRDPTAFSEPESFQPHRWLRNSQPATPRIQHPFGSVPFGYGVRACLGRRIAELEMQLLLARLIQKYKVVLAPETGELKSVARIVLVPNKKVGLQFLQRQC

SEQ ID NO: 2 - DNA, 1596 bp, artificial sequence
  Synthetic Nucleotide sequence encoding CYP27A1
atggctgcgctgggctgcgcgaggctgaggtgggcgctgcgaggggccggccgtggcctctgcccccacggggccagagccaaggccgcgatccctgccgccctcccctcggacaaggccaccggagctcccggagccgggcctggtgtccggcggcggcaacggagcttagaggagattccacgtctaggacagctgcgcttcttctttcagctgttcgttcaaggctatgccctgcaactgcaccagttacaggtgctttacaaggccaagtacggtccaatgtggatgtcctacttagggcctcagatgcacgtgaacctggccagtgccccgctcttggagcaagtgatgcggcaagagggcaagtacccagtacggaacgacatggagctatggaaggagcaccgggaccagcacgacctgacctatgggccgttcaccacggaaggacaccactggtaccagctgcgccaggctctgaaccagcggttgctgaagccagcggaagcagcgctctatacggatgctttcaatgaggtgattgatgactttatgactcgactggaccagctgcgggcagagagtgcttcggggaaccaggtgtcggacatggctcaactcttctactactttgccttggaagctatttgctacatcctgttcgagaaacgcattggctgcctgcagcgatccatccccgaggacaccgtgaccttcgtcagatccatcgggttaatgttccagaactcactctatgccaccttcctccccaagtggactcgccccgtgctgcctttctggaagcgatacctggatggttggaatgccatcttttcctttgggaagaagctgattgatgagaagctcgaagatatggaggcccaactgcaggcagcagggccagatggcatccaggtgtctggctacctgcacttcttactggccagtggacagctcagtcctcgggaggccatgggcagcctgcctgagctgctcatggctggagtggacacgacatccaacacgctgacatgggccctgtaccacctctcaaaggaccctgagatccaggaggccttgcacgaggaagtggtgggtgtggtgccagccgggcaagtgccccagcacaaggactttgcccacatgccgttgctcaaagctgtgcttaaggagactctgcgtctctaccctgtggtccccacaaactcccggatcatagaaaaggaaattgaagttgatggcttcctcttccccaagaacacccagtttgtgttctgccactatgtggtgtcccgggaccccactgccttctctgagcctgaaagcttccagccccaccgctggctgagaaacagccagcctgctacccccaggatccagcacccatttggctctgtgccctttggctatggggtccgggcctgcctgggccgcaggattgcagagctggagatgcagctactcctcgcaaggctgatccagaagtacaaggtggtcctggccccggagacgggggagttgaagagtgtggcccgcattgtcctggttcccaataagaaagtgggcctgcagttcctgcagagacagtgctga

SEQ ID NO: 3 - DNA, 2030 bp, Artificial sequence
  Synthetic CYP27A1 regulatory element
ttaacttttgtgtcaaggcatttttgaacaagcaagggctatccctaaagtcatgaggcagtattgtcctggtgtaccattgggtaatagtagtttcaggattagtctacttgaaaggaaataagttctgagagtcgaagtgcaaaggaatacaaaagaaagcattcttatgatctaacactgtgaactaattggtgttttctggtatttggttaagtatagtataaggatttggcactataggatgtaagagaattatgacctcgttaatggctcctaagtcttgaactagatcatatatatatatatatatatataattttttttttttgagatggagtctcactctgttgtccaggttggagtgcatggcacaatctcagctcactgcaatctccgcctcccgggttcaagcgattctcctgcctcagtctccccaagtagctgggatcacgtgtgccaccacgtttttaaagcatggcctcctgtccttcctgtccaaaaaccaaatgaaaaatatggtctaggttgggtgctgtggctcacacctgtaattccagcactttgggaggccgaggtaggtagatcatctgaggtcaggagtttgagaccagcctggccaacatggcaaaaccctatctctactaaaaatacaaaaattagccgggcgtggtggcgggcacctctaattccagctactctggaggctgagaccggagaattgcttgaaccgggaggcggaggttgcagcgagccaggattgcaccactgcactccagcctggacgacagggtgagactctggggagtggggaggagagaattaacaaactatatgacacaaaggaacaaaaatacacaagacaggataagagagaggaaggatacagggagagctaacatacatatagcagtttgaagtcccgggaaaggagaaaaaaatggggaggagcaatatttgaacagataatatctgagaaactccccagactgatcaaagacatccagtcacagagtcaagaaactaggagtcctagaataaataagtagaaagccacacctagcaactcagctgtcagcctaattgaacttaatatgccctttcttctctggctgcctgtaaatcaccaatttttcaaatgctgttagaaccagctgtaccatcctgctgggtccttcattcctggcttgaatgtgatctttgaccctgtatttattttatacttgcgtgagttatgtttttaaactttttgacaactatactttgaatatacatgaaacataggattcataagacaatactttttttttcttcttgctgtacgttttccgtactatgttactctttccattttattaaaaataattctggccatcatctacgaaatctggtctcaacctgcagtttgaaatatcctgccttggcagacgcggcagggaggtgcgtggagggagaattgaattaaaaaaaaaaacaacaaaacacgaatatttagatttctttgttcagcggcccccctccagggatcagatgactggcccccctcgctccgaactgactccgggatcaatccggaaggccattgggagaagccgagggcagcttagccacggccggttcccgttccctccaggacgcgagggtcgccttgggtggggaaccgcgaccgggcgaggacctatcccggtgtggggcttcccgatttcgaaagaatctcgctgcacccccgcccagagttcagaccaagcgaaaagttatttgagaggcctcgggggcgcggggtgaggagtcgtggcggaggccttggtcggggcgccgtggatatccccgagtcaccgcgtccctctcctgcagctcccgcgtcgctgggaggagcgagggagcgagcgggaaggggtctagctggcctttgctcggccctccccagcgcccggctttgaacccgccctgcactgctgtctgggcgggtccggggactcagcactcgacccaaaggtgcaggcgcgcgagcacaacccgctagcgaattc

SEQ ID NO: 4 - DNA, 305 bp, Artificial sequence
  Synthetic Human alpha-1 anti-trypsin gene promoter
cgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaa

SEQ ID NO: 5 - DNA, 384 bp, Artificial sequence
  Synthetic Mouse serum albumin enhancer
gttcctagattacactacacattctgcaagcatagcacagagcaatgttctactttaattactttcattttcttgtatcctcacagcctagaaaataacctgcgttacagcatccactcagtatcccttgagcatgaggtgacactacttaacatagggacgagatggtactttgtgtctcctgctctgtcagcagggcacagtacttgctgataccagggaatgtttgttcttaaataccatcattccggacgtgtttgccttggccagttttccatgtacatgcagaaagaagtttggactgatcaatacagtcctctgcctttaaagcaataggaaaaggccaacttgtctacgtttagtatgtggctgtagatctgtacc

SEQ ID NO: 6 - DNA, 689 bp, Artificial sequence
  Synthetic Chimeric promoter sequence EATT
gttcctagattacactacacattctgcaagcatagcacagagcaatgttctactttaattactttcattttcttgtatcctcacagcctagaaaataacctgcgttacagcatccactcagtatcccttgagcatgaggtgacactacttaacatagggacgagatggtactttgtgtctcctgctctgtcagcagggcacagtacttgctgataccagggaatgtttgttcttaaataccatcattccggacgtgtttgccttggccagttttccatgtacatgcagaaagaagtttggactgatcaatacagtcctctgcctttaaagcaataggaaaaggccaacttgtctacgtttagtatgtggctgtagatctgtacccgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaa

SEQ ID NO: 7 - DNA, 149 bp, Artificial sequence
  Synthetic 5′-ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgc

SEQ ID NO: 8 - DNA, 148 bp, Artificial Sequence
  Synthetic 3′-ITR
gcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 9 - DNA, 2945 bp, Artificial sequence
  Synthetic Nucleic acid construct ITR-EAAT- CYP27A1-polyA-ITR
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgttcctagattacactacacattctgcaagcatagcacagagcaatgttctactttaattactttcattttcttgtatcctcacagcctagaaaataacctgcgttacagcatccactcagtatcccttgagcatgaggtgacactacttaacatagggacgagatggtactttgtgtctcctgctctgtcagcagggcacagtacttgctgataccagggaatgtttgttcttaaataccatcattccggacgtgtttgccttggccagttttccatgtacatgcagaaagaagtttggactgatcaatacagtcctctgcctttaaagcaataggaaaaggccaacttgtctacgtttagtatgtggctgtagatctgtacccgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaagcggccgctagcgaattcttggcattccggtactgttggtaaagccaccatggctgcgctgggctgcgcgaggctgaggtgggcgctgcgaggggccggccgtggcctctgcccccacggggccagagccaaggccgcgatccctgccgccctcccctcggacaaggccaccggagctcccggagccgggcctggtgtccggcggcggcaacggagcttagaggagattccacgtctaggacagctgcgcttcttctttcagctgttcgttcaaggctatgccctgcaactgcaccagttacaggtgctttacaaggccaagtacggtccaatgtggatgtcctacttagggcctcagatgcacgtgaacctggccagtgccccgctcttggagcaagtgatgcggcaagagggcaagtacccagtacggaacgacatggagctatggaaggagcaccgggaccagcacgacctgacctatgggccgttcaccacggaaggacaccactggtaccagctgcgccaggctctgaaccagcggttgctgaagccagcggaagcagcgctctatacggatgctttcaatgaggtgattgatgactttatgactcgactggaccagctgcgggcagagagtgcttcggggaaccaggtgtcggacatggctcaactcttctactactttgccttggaagctatttgctacatcctgttcgagaaacgcattggctgcctgcagcgatccatccccgaggacaccgtgaccttcgtcagatccatcgggttaatgttccagaactcactctatgccaccttcctccccaagtggactcgccccgtgctgcctttctggaagcgatacctggatggttggaatgccatcttttcctttgggaagaagctgattgatgagaagctcgaagatatggaggcccaactgcaggcagcagggccagatggcatccaggtgtctggctacctgcacttcttactggccagtggacagctcagtcctcgggaggccatgggcagcctgcctgagctgctcatggctggagtggacacgacatccaacacgctgacatgggccctgtaccacctctcaaaggaccctgagatccaggaggccttgcacgaggaagtggtgggtgtggtgccagccgggcaagtgccccagcacaaggactttgcccacatgccgttgctcaaagctgtgcttaaggagactctgcgtctctaccctgtggtccccacaaactcccggatcatagaaaaggaaattgaagttgatggcttcctcttccccaagaacacccagtttgtgttctgccactatgtggtgtcccgggaccccactgccttctctgagcctgaaagcttccagccccaccgctggctgagaaacagccagcctgctacccccaggatccagcacccatttggctctgtgccctttggctatggggtccgggcctgcctgggccgcaggattgcagagctggagatgcagctactcctcgcaaggctgatccagaagtacaaggtggtcctggccccggagacgggggagttgaagagtgtggcccgcattgtcctggttcccaataagaaagtgggcctgcagttcctgcagagacagtgctgagcggccaacaatgcgatccgatggccgcgactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagcccgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcag

SEQ ID NO: 10 - DNA, 21 bp, Artificial sequence
  Synthetic Human Cyp27A1 Forward primer
tgtgcttaaggagactctgcg

SEQ ID NO: 11 - DNA, 22 bp, Artificial sequence
  Synthetic Human Cyp27a1 Reverse primer
atagtggcagaacacaaactgg

SEQ ID NO: 12 - DNA, 22 bp, artificial sequence
  Synthetic Mouse Cyp27a1 exons 1/2 Forward primer
acaaggctatgtgctgcacttg

SEQ ID NO: 13 - DNA, 22 bp, artificial sequence
  Synthetic Mouse Cyp27a1 exons 1/2 reverse primer
tgatccatgtggtctcttattg

SEQ ID NO: 14 - DNA, 20 bp, artificial sequence
  Synthetic Mouse Cyp27a1 exons 8/9 forward primer
atggctgaggaagaaagagg

SEQ ID NO: 15 - DNA, 23 bp, artificial sequence
  Synthetic Mouse Cyp27a1 exons 8/9 reverse primer
acacagtctttacttctcccatc

SEQ ID NO: 16 - DNA, 20 bp, artificial sequence
  Synthetic Mouse Cyp7a1 forward primer
gaagcaatgaaagcagcctc

SEQ ID NO: 17 - DNA, 20 bp, artificial sequence
  Synthetic Mouse Cyp7a1 reverse primer
gtaaatggcattccctccag

SEQ ID NO: 18 - DNA, 27 bp, artificial sequence
  Synthetic Mouse Cyp3a11 forward primer
tgaatatgaaacttgctctcactaaaa

SEQ ID NO: 19 - DNA, 24 bp, artificial sequence
  Synthetic Mouse Cyp3a11reverse primer
ccttgtctgcttaatttcagaggt

SEQ ID NO: 20 - DNA, 21 bp, artificial sequence
  Synthetic Mouse 36b4 forward primer
aacaatctcccccttctcctt

SEQ ID NO: 21 - DNA, 20 bp, artificial sequence
  Synthetic Mouse 36b4 reverse primer
gaaggccttgaccttttcag

(19) United States
(12) Patent Application Publication
(10) Pub. No.: US 2023/0265456 A1
(43) Pub. Date: Aug. 24, 2023

(54) GENE THERAPY VECTOR EXPRESSING CYP27A1 FOR THE TREATMENT OF CEREBROTENDINOUS XANTHOMATOSIS

(71) Applicant: FUNDACION PARA LA INVESTIGACION MEDICA APLICADA, Pamplona (ES)

(72) Inventors: Ruben HERNANDEZ ALCOCEBA, Pamplona (ES); Ana RICOBARAZA ABARQUERO, Alava (ES); Gloria GONZALEZ ASEGUINOLAZA, Baranain (ES)

(21) Appl. No.: 18/041,142

(22) PCT Filed: Feb. 6, 2021
(86) PCT No.: PCT/EP2021/072038

§ 371 Date: Feb. 9, 2023

(30) Foreign Application Priority Data
Aug. 10, 2020 (EP) 20190351.5

(51) Int. Cl. C12N 15/86; A61K 48/00; C12N 5/00

(52) U.S. Cl. CPC C12N 15/86; A61K 48/0058; C12N 5/00; C12N 2750/14143

(57) ABSTRACT

The present disclosure relates to gene therapy vector for use in the treatment of cerebro tendinous xanthomatosis. More specifically, the present invention relates to a nucleic acid construct comprising liver specific promoter operably linked to a nucleic acid sequence encoding for the sterol 27-hydroxylase for the treatment of CTX.

### FIELD OF THE INVENTION

The present invention relates to gene therapy vector for use in the treatment of Cerebrotendinous Xanthomatosis. More specifically, the present invention relates to a nucleic acid construct comprising liver specific promoter operably linked to a nucleic acid sequence encoding for the sterol 27-hydroxylase for the treatment of CTX.

### BACKGROUND ART

Cerebrotendinous Xanthomatosis (CTX) is an autosomal recessive disease caused by mutations in the CYP27A gene with prevalence of 1:50.000-1:100.000 (Lorincz et al. Ardt Neurol. 62 (2005) 1459-1463; Appadurai et al. Mol. Genet. Metab. 116 (2015) 268-304). The causal gene CYP27A1 encodes sterol 27-hydroxylase, a member of cytochrome P450 an enzyme essential for the synthesis of bile acids from cholesterol in the liver. The blockade of this biosynthetic pathway leads to deficits in bile acids (especially chenodeoxycholic acid, CDCA), and accumulation of intermediate metabolites in extrahepatic tissues, such as eyes, tendons and central nervous system (CNS) (Bjeirkhem, Curr. Opin. Lipidol. 24 (2013) 283-287). It is frequent to observe xanthomas, which are deposits of lipids (cholesterol and cholestanol) and reactive cells (Voiculescu et al. J. Neurol. Sci. 82 (1987) 89-99, Pilo De La Fuente et al, J. Neurol. 255 (2008) 839-842). Clinical manifestations include cholestasis, diarrhea, juvenile cataracts, macroscopic xanthomas in joints and tendons, osteoporosis and progressive neurological symptoms (spasticity, ataxia, neuropathy, epilepsy, cognitive and psychiatric alterations). While the neurotoxicity of cholestanol is well stablished, involvement of other metabolites is only suspected (Mignard et al, J. Inherit. Metab. Dis. 39 (2016) 75-83). Treatment with chenodeoxycholic acid (CDCA) compensates the deficiency of endogenous mature species and inhibits de novo synthesis of bile acids, which reduces serum cholestanol levels, whereas other metabolites such as 7-alpha-hydroxy-4-cholesten-3-one (7aC4) are not normalized, as evidenced in the literature (Berginer et al, Pediatrics. 123 (2009) 143-147; Soffer et al. Neuropathol. 90 (1995) 213-20). Thus, it remains a need to develop new treatment that addresses the cause of the disease.

Gene therapy (GT) is the transfer of genetic material to the cells of an organism with a therapeutic purpose. Thanks to the tropism of GT vectors, diseases caused by genetic deficiency in liver enzymes are good candidates for GT, especially if the liver function is not seriously affected, as is the case in CTX. At present, vectors derived from adeno-associated viruses (AAV) are the gold standard for in vivo GT (Li et al. Nat. Reve. Genet. (2020) February 10. doi: 10.1038/s41576-019-0205-4). Animal and clinical studies demonstrated that they can sustain transgene expression for several years (Peyvandi et al. Haemophilia 25 (2019) 738-746). In the design of expression cassettes for gene therapy of monogenic diseases, the best choice is usually the endogenous promoter of the gene being supplemented.

### SUMMARY

Although overexpression of CYP27A1 under the control of ubiquitous chicken 3-actin promoter in transgenic mice did not result in major changes in lipoprotein metabolism (Meir K., et al. (2002) J. Biol. Chem. 277:34036-34041), using a Cypl7al deficient mouse model, the inventors have discovered that the expression of CYP27A1 under the control of a liver-specific promoter is able to correct the metabolic alterations of the disease at relatively low doses. Full correction occurs when only a small percentage of hepatocytes are transduced.

The inventors showed that in contrast to the expression of CYP27A1 under the control of endogenous promoter, a vector encoding the same transgene under the control of a liver-specific promoter is able to correct the metabolic alterations of the disease at relatively low doses. Full correction occurs when only a small percentage of hepatocytes are transduced. This implies that the subset of hepatocytes expressing high levels of CYP27A1 acts as a sink for the toxic metabolites that are accumulated in CTX mice. The expression of CYP27A1 under the control of endogenous promoter has only marginal effect at high doses and would require an extremely high dose of vector to achieve therapeutic efficacy. The vector encoding CYP27A1 under the control of a liver-specific promoter restores bile acid metabolism and normalized the concentration of most bile acids in plasma. In contrast, standard treatment (oral chenodeoxycholic acid, CDCA) while reducing cholestanol, did not normalize bile acid composition in plasma and resulted in supra-physiological levels of CDCA and its derivatives.

A first aspect of the present disclosure thus relates to a nucleic acid construct comprising a liver-specific promoter operably-linked to a transgene encoding human sterol 27-hydroxylase or a variant thereof, preferably said liver-specific promoter comprises a human alpha-1-antitrypsin promoter and/or a mouse albumin enhancer, more preferably a human alpha-1-antitrypsin promoter and a mouse albumin enhancer of SEQ ID NO: 6 or a nucleic acid sequence having at least 80% of identity with SEQ ID NO: 6.

In a particular embodiment, said nucleic acid construct further comprises a 5′ITR and a 3′ITR sequences, preferably a 5′ITR and a 3′ITR sequences of an adeno-associated virus, more preferably a 5′ITR and a 3′ITR sequences from the AAV2 serotype, again more preferably of SEQ ID NO: 7 and 8. In a preferred embodiment, said nucleic acid construct comprises nucleic acid sequence SEQ ID NO: 9 or a nucleic acid sequence having at least 80% of identity with SEQ ID NO: 9.

In another aspect, the present invention relates to an expression vector, preferably a viral vector, more preferably an adeno-associated viral (AAV) vector comprising said nucleic acid construct.

The present invention also relates to a viral particle, preferably an AAV particle comprising said nucleic acid construct or expression vector, and more preferably comprising capsid proteins of adeno-associated virus such as capsid proteins selected from the group consisting of: AAV3 type 3A, AAV3 type 3B, NP40, NP59, NP84, LK03, AAV3-ST, Anc80, AAV9 and AAV8 serotype.

In another aspect, the present invention relates to a host cell comprising said nucleic acid construct or expression vector or a host cell transduced with a viral particle as described above.

The present invention also relates to a pharmaceutical composition comprising said nucleic acid construct, vector, viral particle or host cell and a pharmaceutically acceptable excipient.

In another aspect, the present invention relates to said nucleic acid construct, vector, viral particle, host cell or pharmaceutical composition for its use as a medicament in a subject in need thereof, preferably for the prevention and/or treatment of Cerebrotendinous Xanthomatosis (CTX) in a subject in need thereof.

Finally, the present invention relates to a method of producing viral particles as described above, comprising the steps of:
a) culturing a host cell comprising said nucleic acid construct or expression vector in a culture medium, and
b) harvesting the viral particles from the cell culture supernatant and/or inside the cells,
c) optionally purifying and formulating said viral particles.

### BRIEF DESCRIPTION OF THE DRAWINGS

FIG. 1. Schematic representation of vector genomes. In both cases, the transgene is the human CYP27A1 cDNA. EAAT is a hybrid promoter consisting of the mouse albumin enhancer and the human alpha 1 anti-trypsin promoter. C27P is the 2 Kb 5′UTR fragment from the human CYP27A1 gene. ITR, inverted terminal repeats from AAV2; pA, polyadenylation signal. Genomes are packaged in AAV8 capsids.

FIG. 2. Evaluation of promoters in vivo. Plasmids expressing the luciferase reporter gene under the control of the indicated promoters (left pictures) were administered to C57BL/6 mice by hydrodynamic injection. Forty-eight hours later, mice received an intraperitoneal injection of the luciferase substrate luciferin, and light mission was quantified using an in vivo luciferase imaging system. The values represented in the graph correspond to photons/second emitted from the hepatic region. *p<0.05, ANOVA.

FIG. 3. Metabolic correction of Cyp27a k.o. mice treated with AAV8-EAAT-CYP27A1. Six weeks-old Cyp27a1 k.o. mice were treated with with 1.5×10¹² or 1.5×10¹³ vg/kg of the vector by intravenous injection. Two weeks later, blood was collected for determination of cholestanol (A) and 7-alpha-hydroxy-4-cholesten-3-one (7aC4) (B) in plasma. Wild-type C57BL/6 mice (WT) are included as a reference for normal values of each metabolite. *p<0.05; ***p<0.001, ANOVA

FIG. 4. Inefficient metabolic correction of Cyp27a k.o. mice treated with AAV8-C27P-CYP27A1. Six weeks-old Cyp27a) k.o. mice were treated with 5×10¹² or 1.5×10¹³ vg/kg of the vector by intravenous injection. Two weeks later, blood was collected for determination of cholestanol (A) and 7-alpha-hydroxy-4-cholesten-3-one (7aC4) (B) in plasma. Wild type C57BL/6 mice (WT) are included as a reference for normal values of each metabolite. *p<0.05; ***p<0.001, ANOVA.

FIG. 5. Expression of CYP27A1 in the liver of mice treated with the AAV vectors. Six weeks-old Cyp27a1 k.o. mice were treated with AAV8-EAAT-CYP27A1 (EAAT) or AAV8-C27P-CYP27A1 (C27P) vectors by intravenous injection at the indicated doses (in vg/kg). Two weeks later mice were sacrificed, and livers were collected for analysis of CYP27A1 content by immunohistochemistry. Control is a representative untreated Cyp27a1 k.o. mice. Positive cells can be distinguished by darker staining.

FIG. 6. The EAAT promoter shows stronger activity than the CYP27A1 5′UTR in some cell limes and in vivo. A. Schematic representation of luciferase reporter plasmids containing the CMV promoter, the hybrid liver-specific promoter EAAT (albumin enhancer linked to the al anti-trypsin promoter) and the 5′UTR from the human CYP27A1 gene (C27P). The promoterless plasmid pGL3-Basic was used as a negative control. B. The plasmids were transfected in the indicated liver-derived cell lines from human (HuH-7, HepG2 and Hep3B) and mouse (Hepal-6 and AML12) origin. Luciferase activity was measured in cell extracts obtained 48 hours after transfection. The result is expressed as percentage of activity, considering the CMV promoter as a reference. C. The plasmids were injected in C57BL/6 mice (n=6) by hydrodynamic injection, and light emission was quantified by BLI 48 hours later. *p<0.05, Kruskal-Wallis.

FIG. 7. Expression of CYP27A1 from AAV vectors. A. Schematic representation of vector genomes. ITR, inverted terminal repeat; pA, polyadenylation signal. B. The AAV8-EAAT-CYP27A1 and AAV8-C27P-CYP27A1 vectors (abbreviated as EAAT and C27P, respectively) were administered intravenously to 7 week-old CTX mice at the indicated doses, and 2 weeks later mice were sacrificed for quantification of human and mouse CYP27A1 mRNA by qRT-PCR. WT littermates were included as a reference. Data are represented as relative mRNA content, using the housekeeping gene 36b4 as a reference and multiplied by a factor of 1.000 for easier visualization (WT and CTX controls n=10; treated CTX n=5). Data include pooled male and female mice because no difference was observed between both genders. C. One portion of liver samples was used to detect the CYP27A1 protein by Western blot. Image shows a representative blot together with quantification of all samples (WT and CTX controls n=10; treated CTX n=5). Note that the antibody is able to detect both mouse and human protein using this technique. D. Immunohistochemistry for detection of CYP27A1 (representative images), and quantification of positive hepatocytes. *p<0.05; ***p<0.001; ****p<0.0001. Kruskal-Wallis with Dunn's post-test.

FIG. 8. AAV8-EAAT-CYP27A1 and CDCA reduce cholestanol and 7aC4 in CTX mice. The AAV8-EAAT-CYP27A1 or AAV8-C27P-CYP27A1 vectors were administered intravenously to 7 week-old CTX mice at the indicated doses. CDCA was mixed in mouse chow at 0.1, 0.5 or 1%. Blood was collected 2 weeks after vector administration, or 1 month after initiation of CDCA diet. The concentration of cholestanol and 7aC4 was determined in mouse plasma. Untreated CTX mice and WT littermates were included as a reference (WT n=10; CTX n=15; treated CTX n=5 each gender). *p<0.05; ″p<0.01; ***p<0.001 versus untreated CTX mice. Kruskal-Wallis with Dunn's post-test.

FIG. 9. AAV8-EAAT-CYP27A1 is well-tolerated and achieves sustained therapeutic effect after a single administration. The AAV8-EAAT-CYP27Alvector (EAAT) was administered intravenously to 7 week-old CTX mice at the indicated doses (×10¹² vg/Kg). A. Some mice from each group (n=4) were sacrificed 15 days after vector administration, and the rest (n=7) were maintained for 5 months. CYP27A1 mRNA was measured by qRT-PCR in liver samples. Data are represented as relative mRNA content, using the housekeeping gene 36b4 as a reference and multiplied by a factor of 1.000 for easier visualization. Blood samples were obtained at the indicated times for measurement of cholestanol and 7aC4 (B) and the transaminase ALT (C). Untreated CTX mice and WT littermates were included as a reference. Data corresponds to equilibrated groups of female and male mice. D. Representative images of liver histology (Hematoxylin & Eosin staining) of mice 5 months after initiation of treatment. *p<0.05; ***p<0.001 versus untreated CTX mice. Kruskal-Wallis with Dunn's post-test.

FIG. 10. AAV8-EAAT-CYP27A1, but not CDCA, normalizes the expression of Cyp7a1 and Cyp3a1 1 in CTX mice. The AAV8-EAAT-CYP27A1 or AAV8-C27P-CYP27A1 vectors were administered intravenously to 7 week-old CTX mice at the indicated doses. CDCA was mixed in mouse chow at 0.1 or 0.5%. Untreated CTX and WT littermates were included as a reference. Animals were sacrificed 2 weeks after vector administration, or 1 month after initiation of CDCA diet, and liver samples were obtained for quantification of endogenous Cyp27a1 (A), Cyp7a1 (B) and Cyp3a11 (C) expression. Data are represented as relative mRNA content, using the housekeeping gene 36b4 as a reference and multiplied by a factor of 1.000 for easier 839 visualization (WT and CTX control n=15; EAAT and C27P n=5; CDCA n=8). *p<0.05; **p<0.01; ***p<0.001 versus untreated CTX mice. Kruskal-Wallis with Dunn's post-test.

FIG. 11. AAV8-EAAT-CYP27A1 reverses hepatomegaly in CTX mice. The AAV8-EAAT-CYP27A1 vector was administered intravenously to 7 week-old CTX mice at the indicated doses. CDCA was mixed in mouse chow at 0.1 or 0.5%. Untreated CTX and WT littermates were included as a reference. Mice were maintained for 3 months and then they were sacrificed for determination of relative liver weight, represented as percentage of body weight. *p<0.05; ***p<0.001 versus untreated CTX mice. Kruskal-Wallis with Dunn's post-test.

FIG. 12. AAV8-EAAT-CYP27A1, but not CDCA, normalizes bile acid composition in blood of CTX mice. The AAV8-EAAT-CYP27A1 vector was administered intravenously to 7 week-old CTX mice at the indicated doses (×10¹² vg/Kg). CDCA was mixed in mouse chow at 0.5%. Untreated CTX and WT littermates were included as a reference. Blood was collected 5 months after the initiation of treatment and the main free bile acids and tauroconjugates were analysed in plasma. CDCA, chenodeoxycholic acid; aMCA, α-muricholic acid; bMCA, β-muricholic acid; LCA, litocholic acid; UDCA, ursodeoxycholic acid; HDCA, hyodeoxycholic acid; TDCA, taurodeoxycholic acid; TMCA, tauromuricholic acid; TLCA, taurolitocholic acid; TUDCA, tauroursodeoxycholic acid; THDCA, taurohyodeoxycholic acid; CA, cholic acid; DCA, deoxycholic acid; TCA, taurocholic acid; TDCA, taurodeoxycholic acid. *p<0.05; **p<0.01; ***p<0.001. Kruskal-Wallis with Dunn's post-test.

### DETAILED DESCRIPTION

The present disclosure relates to a nucleic acid construct comprising a transgene encoding a human sterol 27-hydroxylase also called sterol 26-hydroxylase, mitochondrial precursor (NCBI reference Sequence: NP_000775.1 accessed on Apr. 25, 2020) (SEQ ID NO: 1) or a variant thereof.

The cytochrome P450 proteins are monooxygenases which catalyze many reactions involved in drug metabolism and synthesis of cholesterol, steroids and other lipids. This mitochondrial protein oxidizes cholesterol intermediates as part of the bile synthesis pathway.

CYP27A1 protein is encoded by the cytochrome P450 family 27 subfamily A member 1, Sterol 27 (CYP27A1) gene (Gene ID: 1593 accessed on Jun. 4, 2020) also called CTX, CP27 or CYP27 hydroxylase (CYP27).

As used herein, the term “transgene” refers to exogenous DNA or cDNA encoding a gene product. The gene product may be an RNA, peptide or protein. In addition to the coding region for the gene product, the transgene may include or be associated with one or more elements to facilitate or enhance expression, such as a promoter, enhancer(s), response element(s), reporter element(s), insulator element(s), polyadenylation signal(s) and/or other functional elements. Embodiments of the disclosure may utilize any known suitable promoter, enhancer(s), response element(s), reporter element(s), insulator element(s), polyadenylation signal(s) and/or other functional elements. Suitable elements and sequences will be well known to those skilled in the art.

The terms “nucleic acid sequence” and “nucleotide sequence” may be used interchangeably to refer to any molecule composed of or comprising monomeric nucleotides. A nucleic acid may be an oligonucleotide or a polynucleotide. A nucleotide sequence may be a DNA or RNA. A nucleotide sequence may be chemically modified or artificial. Nucleotide sequences include peptide nucleic acids (PNA), morpholinos and locked nucleic acids (LNA), as well as glycol nucleic acids (GNA) and threose nucleic acid (TNA). Each of these sequences is distinguished from naturally-occurring DNA or RNA by changes to the backbone of the molecule. Also, phosphorothioate nucleotides may be used. Other deoxynucleotide analogs include methylphosphonates, phosphoramidates, phosphorodithioates, N3′P5′-phosphoramidates and oligoribonucleotide phosphorothioates and their 2′-O-allyl analogs and 2′-O-methylribonucleotide methylphosphonates which may be used in a nucleotide of the disclosure.

The transgene according to the disclosure may be any nucleic acid sequence encoding a sterol 27-hydroxylase, in particular a native mammalian, preferably human sterol 27-hydroxylase (SEQ ID NO: 1) or a functional variant thereof.

Preferably, as used herein, the term “variant” or “functional variant” refers to a polypeptide having an amino acid sequence having at least 70, 75, 80, 85, 90, 95 or 99% sequence identity to the native sequence and retain function of said polypeptide, herein enzymatic function of sterol 27-hydroxylase.

As used herein, the term “sequence identity” or “identity” refers to the number (%) of matches (identical amino acid residues) in positions from an alignment of two polynucleotide or polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithms (e.g. Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al, 1997; Altschul et al., 2005). Alignment for purposes of determining percent nucleic acid or amino acid sequence identity can be achieved in various ways that are within the skill in the art, for instance, using publicly available computer software available on internet web sites such as http://blast.ncbi.nlm.nih.gov/ or http://www.ebi.ac.uk/Tools/emboss/. Those skilled in the art can determine appropriate parameters for measuring alignment, including any algorithms needed to achieve maximal alignment over the full length of the sequences being compared. For purposes herein, % nucleic acid or amino acid sequence identity values refers to values generated using the pair wise sequence alignment program EMBOSS Needle that creates an optimal global alignment of two sequences using the Needleman-Wunsch algorithm, wherein all search parameters are set to default values, i.e. Scoring matrix=BLOSUM62, Gap open=10, Gap extend=0.5, End gap penalty=false, End gap open=10 and End gap extend=0.5.

More preferably, the term “variant” or “functional variant” refers to a polypeptide having an amino acid sequence that differs from a native sequence by less than 30, 25, 20, 15, 10 or 5 substitutions, insertions and/or deletions. In a preferred embodiment, the variant differs from the native sequence by one or more conservative substitutions, preferably by less than 15, 10 or 5 conservative substitutions. Examples of conservative substitutions are within the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine), and small amino acids (glycine, alanine, serine and threonine). Sterol 27-hydroxylase activity of a variant may be assessed by any method known by the skilled person, for instance by measuring the enzymatic activity of said sterol 27-hydroxylase variant.

In specific embodiment, the present disclosure relates to a nucleic acid construct comprising a transgene encoding human sterol 27-hydroxylase, said transgene is a mammalian coding sequence of sterol 27-hydroxylase, preferably a human coding sequence of sterol 27-hydroxylase (NCBI Reference Sequence: NM_000784.4 accessed on Apr. 25, 2020), preferably of SEQ ID NO: 2 or a nucleic acid sequence having at least 70, 75, 80, 85, 90, 95 or 99% sequence identity with SEQ ID NO: 2.

The coding sequences of a number of different mammalian sterol 27-hydroxylase are known including, but being not limited to, human, pig, chimpanzee, dog, cow, mouse, rabbit or rat, and can be easily found in sequence databases. Alternatively, the coding sequence may be easily determined by the skilled person based on the polypeptide sequence.

In another particular embodiment said transgene may be an optimized sequence encoding sterol 27-hydroxylase or variant thereof.

The term “codon optimized” means that a codon that expresses a bias for human (i.e. is common in human genes but uncommon in other mammalian genes or non-mammalian genes) is changed to a synonymous codon (a codon that codes for the same amino acid) that does not express a bias for human. Thus, the change in codon does not result in any amino acid change in the encoded protein.

Nucleic Acid Construct

The term “nucleic acid construct” as used herein refers to a man-made nucleic acid molecule resulting from the use of recombinant DNA technology. A nucleic acid construct is a nucleic acid molecule, either single- or double-stranded, which has been modified to contain segments of nucleic acids sequences, which are combined and juxtaposed in a manner, which would not otherwise exist in nature. A nucleic acid construct usually is a “vector”, i.e. a nucleic acid molecule which is used to deliver exogenously created DNA into a host cell.

Said nucleic acid construct comprises one or more control sequence required for expression of said coding sequence. Generally, the nucleic acid construct comprises a coding sequence and regulatory sequences preceding (5′ non-coding sequences) and following (3′ non-coding sequences) the coding sequence that are required for expression of the selected gene product. Thus, a nucleic acid construct typically comprises a promoter sequence, a coding sequence and a 3′ untranslated region that usually contains a polyadenylation site and/or transcription terminator. The nucleic acid construct may also comprise additional regulatory elements such as, for example, enhancer sequences, a polylinker sequence facilitating the insertion of a DNA fragment within a vector and/or splicing signal sequences.

According to the present disclosure, said nucleic acid construct comprises liver-specific regulatory elements, preferably strong liver-specific regulatory elements operably linked to a transgene encoding sterol 27-hydroxylase.

In particular embodiment, said regulatory element comprises a promoter that initiates transgene expression upon introduction into a host cell. As used herein, the term “promoter” refers to a regulatory element that directs the transcription of a nucleic acid to which it is operably linked. A promoter can regulate both rate and efficiency of transcription of an operably linked nucleic acid. A promoter may also be operably linked to other regulatory elements which enhance (“enhancers”) or repress (“repressors”) promoter-dependent transcription of a nucleic acid. These regulatory elements include, without limitation, transcription factor binding sites, repressor and activator protein binding sites, and any other sequences of nucleotides known to one of skill in the art to act directly or indirectly to regulate the amount of transcription from the promoter, including e.g. attenuators, enhancers, and silencers. The promoter is located near the transcription start site of the gene or coding sequence to which it is operably linked, on the same strand and upstream of the DNA sequence (towards the 5′ region of the sense strand). A promoter can be about 100-3000 base pairs long. Positions in a promoter are designated relative to the transcriptional start site for a particular gene (i.e., positions upstream are negative numbers counting back from −1, for example −100 is a position 100 base pairs upstream).

As used herein, the term “operably linked” refers to a linkage of polynucleotide (or polypeptide) elements in a functional relationship. A nucleic acid is “operably linked” when it is placed into a functional relationship with another nucleic acid sequence. For instance, a promoter or transcription regulatory sequence is operably linked to a coding sequence if it affects the transcription of the coding sequence. Operably linked means that the DNA sequences being linked are typically but not necessarily contiguous; where it is necessary to join two protein encoding regions, they are contiguous and in reading frame.

In the context of this disclosure, a “liver-specific promoter” is a promoter which is more active in the liver than in any other tissue of the body. Typically, the activity of a liver specific promoter will be considerably greater in the liver than in other tissues. For example, such a promoter may be at least 2, at least 3, at least 4, at least 5 or at least 10 times more active (for example as determined by its ability to drive the expression in a given tissue in comparison to its ability to drive the expression in other cells or tissues). Accordingly, a liver-specific promoter allows an active expression in the liver of the gene linked to it and prevents its expression in other cells or tissues.

In the context of the disclosure, a “strong promoter” is a promoter which is more active than the endogenous promoter of said transgene. According to the present disclosure, the activity of a strong promoter may be at least 2, at least 3, at least 4, at least 5 or at least 10 times more active (for example as determined by its ability to drive the expression of the transgene in a given tissue in comparison to the ability to drive the expression of the same transgene inserted downstream endogenous regulatory element in the same tissue). According to the present disclosure said endogenous promoter is CYP27A1 regulatory elements, in particular of SEQ ID NO: 3.

To test promoter activity, the promoter may be operably linked to a screenable marker and introduced into a host cell. The expression level of the screenable marker may be assessed and the promoter activity may be determined based on the level of expression of the screenable marker. The biological activity of the promoter may be determined either visually or quantitatively based on levels of screenable marker expression in host cells.

In a particular embodiment, said liver-specific promoter may be a strong liver-specific promoter selected in the group consisting of: α1-antitrypsin gene promoter (AAT or A1AT), bile salt-inducible promoter, albumin, hemopexin, transtyretin, phosphoglycerate kinase, preferably human α1-antitrypsin gene promoter of SEQ ID NO: 4 or a sequence having at least 70, 75, 80, 85, 90, 95 or 99% of identity with SEQ ID NO: 4.

The liver-specific promoter according to the disclosure may further comprises a liver-specific enhancer elements that is capable of enhancing liver-specific expression of the transgene in the liver.

Such liver-specific enhancers include one or more serum albumin enhancers, prothrombin enhancers, α-I microglobulin enhancers and an intronic aldolase enhancers, preferably mouse serum albumin enhancer of SEQ ID NO: 5 or a sequence having 70, 75, 80, 85, 90, 95 or 99% with SEQ ID NO: 5.

In a more preferred embodiment, said liver-specific promoter is a strong liver-specific promoter such as chimeric promoter sequence EalbPa1AT (EAAT) that comprises a human α1-antitrypsin gene promoter sequence (AAT or Pa1AT) combined with a mouse albumin gene enhancer element (Ealb), preferably of SEQ ID NO: 6 or a sequence having at least 70, 75, 80, 85, 90, 95 or 99% of identity with SEQ ID NO: 6.

Each of these nucleic acid construct embodiments may also include a polyadenylation signal sequence; together or not with other optional nucleotide elements. As used herein, the term “polyadenylation signal” or “poly(A) signal” refers to a specific recognition sequence within 3′ untranslated region (3′ UTR) of the gene, which is transcribed into precursor mRNA molecule and guides the termination of the gene transcription. Poly(A) signal acts as a signal for the endonucleolytic cleavage of the newly formed precursor mRNA at its 3′-end, and for the addition to this 3′-end of a RNA stretch consisting only of adenine bases (polyadenylation process; poly(A) tail). Poly(A) tail is important for the nuclear export, translation, and stability of mRNA. In the context of the disclosure, the polyadenylation signal is a recognition sequence that can direct polyadenylation of mammalian genes and/or viral genes, in mammalian cells.

Poly(A) signals typically consist of a) a consensus sequence AAUAAA, which has been shown to be required for both 3′-end cleavage and polyadenylation of premessenger RNA (pre-mRNA) as well as to promote downstream transcriptional termination, and b) additional elements upstream and downstream of AAUAAA that control the efficiency of utilization of AAUAAA as a poly(A) signal. There is considerable variability in these motifs in mammalian genes.

In one embodiment, the polyadenylation signal sequence of the nucleic acid construct of the disclosure is a polyadenylation signal sequence of a mammalian gene or a viral gene. Suitable polyadenylation signals include, among others, a SV40 early polyadenylation signal, a SV40 late polyadenylation signal, a HSV thymidine kinase polyadenylation signal, a protamine gene polyadenylation signal, an adenovirus 5 EIb polyadenylation signal, a growth hormone polydenylation signal, a PBGD polyadenylation signal, in silico designed polyadenylation signal (synthetic) and the like.

In a particular embodiment, the polyadenylation signal sequence of the nucleic acid construct is a synthetic poly(A) signal sequence based on the SV40 late polyA gene.

Expression Vector

The nucleic acid construct of the disclosure may be comprised in an expression vector. As used herein, the term “expression vector” refers to a nucleic acid molecule used as a vehicle to transfer genetic material, and in particular to deliver a nucleic acid into a host cell, either in vitro or in vivo. Expression vector also refers to a nucleic acid molecule capable of effecting expression of a gene (transgene) in host cells or host organisms compatible with such sequences. Expression vectors typically include at least suitable transcription regulatory sequences and optionally 3′-transcription termination signals. Additional factors necessary or helpful in effecting expression may also be present, such as expression enhancer elements able to respond to a precise inductive signal (endogenous or chimeric transcription factors) or specific for certain cells, organs or tissues. Vectors include, but are not limited to, plasmids, phasmids, cosmids, transposable elements, viruses, and artificial chromosomes (e.g., YACs). Preferably, the vector of the disclosure is a vector suitable for use in gene or cell therapy, and in particular is suitable to target liver cells.

In some embodiments, the expression vector is a viral vector, such as vectors derived from Moloney murine leukemia virus vectors (MoMLV), MSCV, SFFV, MPSV or SNV, lentiviral vectors (e.g. derived from human immunodeficiency virus (HIV), simian immunodeficiency virus (SIV), feline immunodeficiency virus (FIV), bovine immunodeficiency virus (BIV) or equine infectious anemia virus (EIAV)), adenoviral (Ad) vectors, adeno-associated viral (AAV) vectors, simian virus 40 (SV-40) vectors, bovine papilloma virus vectors, Epstein-Barr virus, herpes virus vectors, vaccinia virus vectors, Harvey murine sarcoma virus vectors, murine mammary tumor virus vectors, Rous sarcoma virus vectors.

As is known in the art, depending on the specific viral vector considered for use, suitable sequences should be introduced in the vector of the disclosure for obtaining a functional viral vector, such as AAV ITRs for an AAV vector, or LTRs for lentiviral vectors. In a particular embodiment, said vector is an AAV vector.

AAV has arisen considerable interest as a potential vector for human gene therapy. Among the favourable properties of the virus are its lack of association with any human disease, its ability to infect both dividing and non-dividing cells, and the wide range of cell lines derived from different tissues that can be infected. The AAV genome is composed of a linear, single-stranded DNA molecule which contains 4681 bases (Berns and Bohenzky, 1987, Advances in Virus Research (Academic Press, Inc.) 32:243-307). The genome includes inverted terminal repeats (ITRs) at each end, which function in cis as origins of DNA replication and as packaging signals for the virus. The ITRs are approximately 145 bp in length. The internal non-repeated portion of the genome includes two large open reading frames, known as the AAV rep and cap genes, respectively. These genes code for the viral proteins involved in replication and packaging of the virion. In particular, at least four viral proteins are synthesized from the AAV rep gene, Rep 78, Rep 68, Rep 52 and Rep 40, named according to their apparent molecular weight. The AAV cap gene encodes at least three proteins, VP1, VP2 and VP3. For a detailed description of the AAV genome, see, e.g., Muzyczka, N. 1992 Current Topics in Microbiol. and Immunol. 158:97-129.

Thus, in one embodiment, the nucleic acid construct or expression vector comprising transgene of the disclosure further comprises a 5′ITR and a 3′ITR sequences, preferably a 5′ITR and a 3′ ITR sequences of an adeno-associated virus.

As used herein the term “inverted terminal repeat (ITR)” refers to a nucleotide sequence located at the 5′-end (5′ITR) and a nucleotide sequence located at the 3′-end (3′ITR) of a virus, that contain palindromic sequences and that can fold over to form T-shaped hairpin structures that function as primers during initiation of DNA replication. They are also needed for viral genome integration into the host genome; for the rescue from the host genome; and for the encapsidation of viral nucleic acid into mature virions. The ITRs are required in cis for the vector genome replication and its packaging into the viral particles.

AAV ITRs for use in the viral vector of the disclosure may have a wild-type nucleotide sequence or may be altered by the insertion, deletion or substitution. The serotype of the inverted terminal repeats (ITRs) of the AAV may be selected from any known human or nonhuman AAV serotype. In specific embodiments, the nucleic acid construct or viral expression vector may be carried out by using ITRs of any AAV serotype, including AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV, and any other AAV serotype now known or later discovered.

In one embodiment, the nucleic acid construct can be designed to be self-complementary AAV (scAAV). “Self-complementary AAV” refers to AAV vector designed to form an intra-molecular double-stranded DNA template which does not require DNA synthesis (D M McCarty et al. 2001. Gene Therapy, 8(16):1248-1254). Upon infection, rather than waiting for cell mediated synthesis of the second strand, the two complementary halves of scAAV will associate to form one double stranded DNA (dsDNA) unit that is ready for immediate replication and transcription. For example, the AAV may be engineered to have a genome comprising two connected single-stranded DNAs that encode, respectively, a transgene unit and its complement, which can snap together following delivery into a target cell, yielding a double-stranded DNA encoding the transgene unit of interest. Self-complementary AAVs are described in for instance U.S. Pat. Nos. 6,596,535; 7,125,717 and 7,456,683.

In one embodiment, the nucleic acid construct further comprises a 5′ITR and a 3′ITR of an AAV of a serotype AAV2, preferably of SEQ ID NO: 7 and 8.

In a particular embodiment, the nucleic acid construct of the disclosure comprises or consists of SEQ ID NO: 9 or a sequence having at least 70, 75, 80, 85, 90, 95 or 99% of identity with SEQ ID NO: 9.

In one embodiment, the nucleic acid construct or AAV vector genome according to the disclosure is comprised in a recombinant baculovirus genome. As used herein, the term “recombinant baculovirus genome” refers to a nucleic acid that comprises baculoviral genetic elements for autonomous replication of a recombinant baculovirus genome in a host cell permissive for baculovirus infection and replication, typically insect cells. The term “recombinant baculovirus genome” expressly includes genomes comprising nucleic acids that are heterologous to the baculovirus. Likewise, the term “recombinant baculovirus genome” does not necessarily refer to a complete baculovirus genome as the genome may lack viral sequences that are not necessary for completion of an infection cycle. In particular, the recombinant baculovirus genomes may include the heterologous AAV genes useful for rAAV production and/or the transgene such as sterol 27-hydroxylase cDNA to be encapsidated in the rAAV for use in gene therapy. The baculoviral genetic elements for use in the present disclosure are preferably obtained from AcMNPV baculovirus (Autographa californica multinucleocapsid nucleopolyhedrovirus).

In a particular embodiment, the genes encoding baculovirus cathepsin and chitinase in said first and second baculoviral genomes are disrupted or deleted. In particular, the genes v-cath (Ac127) and chiA (Ac126) of the AcMNPV baculovirus may be disrupted or deleted so that the corresponding cathepsin or chitinase are either not expressed or expressed as inactive forms (i.e. have no enzymatic cathepsin or chitinase activity). In a particular embodiment, said recombinant baculovirus genomes are further disrupted or deleted for at least p24 gene (Ac129), preferably for the three baculoviral genes p10 (Ac137), p24 and p26 (Ac136). In a particular embodiment, said recombinant baculovirus genomes include functional p74 baculoviral gene (Ac138) (i.e. said gene has not been deleted or disrupted).

On the other hand, the nucleic acid construct or expression vector of the disclosure may be carried out by using synthetic 5′ITR and/or 3′ITR; and also by using a 5′ITR and a 3′ITR which come from viruses of different serotypes. All other viral genes required for viral vector replication can be provided in trans within the virus-producing cells (packaging cells) as described below. Therefore, their inclusion in the viral vector is optional.

In one embodiment, the nucleic acid construct or viral vector of the disclosure comprises a 5′ITR, a ψ packaging signal, and a 3′ITR of a virus. “ψ packaging signal” is a cis-acting nucleotide sequence of the virus genome, which in some viruses (e.g. adenoviruses, lentiviruses . . . ) is essential for the process of packaging the virus genome into the viral capsid during replication.

The construction of recombinant AAV viral particles is generally known in the art and has been described for instance in U.S. Pat. Nos. 5,173,414 and 5,139,941; WO 92/01070, WO 93/03769, Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801.

Viral Particle

The nucleic acid construct or the expression vector of the disclosure may be packaged into a virus capsid to generate a “viral particle”, also named “viral vector particle”. In a particular embodiment, the nucleic acid construct or the expression vector of the disclosure is packaged into an AAV-derived capsid to generate an “adeno-associated viral particle” or “AAV particle”. The present disclosure relates to a viral particle comprising a nucleic acid construct or an expression vector of the disclosure and preferably comprising capsid proteins of adeno-associated virus.

The term AAV vector particle encompasses any recombinant AAV vector particle or mutant AAV vector particle, genetically engineered. A recombinant AAV particle may be prepared by encapsidating the nucleic acid construct or viral expression vector including ITR(s) derived from a particular AAV serotype in a viral particle formed by natural or mutant Cap proteins corresponding to an AAV of the same or different serotype.

Proteins of the viral capsid of an adeno-associated virus include the capsid proteins VP1, VP2, and VP3. Differences among the capsid protein sequences of the various AAV serotypes result in the use of different cell surface receptors for cell entry. In combination with alternative intracellular processing pathways, this gives rise to distinct tissue tropisms for each AAV serotype.

Several techniques have been developed to modify and improve the structural and functional properties of naturally occurring AAV viral particles (Bunning H et al. J Gene Med, 2008; 10: 717-733; Paulk et al. Mol ther. 2018; 26(1):289-303; Wang L et al. Mol Ther. 2015; 23(12):1877-87; Vercauteren et al. Mol Ther. 2016; 24(6):1042-1049; Zinn E et al., Cell Rep. 2015; 12(6):1056-68).

Thus, in AAV viral particle according to the present disclosure, the nucleic acid construct or viral expression vector including ITR(s) of a given AAV serotype can be packaged, for example, into: a) a viral particle constituted of capsid proteins derived from the same or different AAV serotype [e.g. AAV2 ITRs and AAV5 capsid proteins; AAV2 ITRs and AAV8 capsid proteins; AAV2 ITRs and Anc80 capsid proteins; AAV2 ITRs and AAV9 capsid proteins]; b) a mosaic viral particle constituted of a mixture of capsid proteins from different AAV serotypes or mutants [e.g. AAV2 ITRs with AAV1 and AAV5 capsid proteins]; c) a chimeric viral particle constituted of capsid proteins that have been truncated by domain swapping between different AAV serotypes or variants [e.g. AAV2 ITRs with AAV5 capsid proteins with AAV3 domains].

The skilled person will appreciate that the AAV viral particle for use according to the present disclosure may comprise capsid proteins from any AAV serotype including AAV1, AAV2, AAV3 (including types 3A and 3B), AAV4, AAV5, AAV6, AAV7, AAV8, AAV9, AAV10, AAV11, AAV12, avian AAV, bovine AAV, canine AAV, equine AAV, ovine AAV, synthetic AAV variants such as NP40, NP59, NP84 (Paulk et al. Mol ther. 2018.26(1):289-303), LK03 (Wang L et al. Mol Ther. 2015. 23(12):1877-87), AAV3-ST (Vercauteren et al. Mol Ther. 2016.24(6):1042-1049), Anc80 (Zinn E et al., Cell Rep. 2015; 12(6):1056-68) and any other AAV serotype now known or later discovered.

In a specific embodiment, the AAV viral particle comprises capsid proteins from a serotype selected from the group consisting of an AAV1, AAV3B, an AAV5, an AAV7, an AAV8, and an AAV9 which are more suitable for delivery to the liver cells (Nathwani et al. Blood 2007; 109: 1414-1421; Kitajima et al. Atherosclerosis 2006; 186:65-73).

In a particular embodiment, the AAV viral particle comprises capsid proteins from Anc80, a predicted ancestor of viral AAVs serotypes 1, 2, 8, and 9 that behaves as a highly potent gene therapy vector for targeting liver, muscle and retina (Zinn E et al., Cell Rep. 2015; 12(6):1056-68). In a more particular embodiment, the viral particle comprises the Anc80L65 VP3 capsid protein (Genbank accession number: KT235804).

Thus, in a further aspect, the present disclosure relates to a viral particle comprising a nucleic acid construct or expression vector of the disclosure and preferably comprising capsid proteins of adeno-associated virus such as capsid proteins are selected from the group consisting of: AAV3 type 3A, AAV3 type 3B, NP40, NP59, NP84, LK03, AAV3-ST, Anc80, AAV9 and AAV8 serotype.

In a particular embodiment, the viral particle comprises AAV vector genome comprised in recombinant baculovirus. Thus, a second recombinant baculovirus genome comprising AAV rep and cap is used for producing AAV viral particle. In a particular embodiment, the rep and cap proteins are expressed from distinct baculovirus late promoters, preferably in inverse orientation. In a specific embodiment, that may be combined with the previous embodiments, the second baculovirus genome include a heterologous nucleic acid encoding the rep proteins, for example, rep proteins from AAV2 under the transcriptional control of the baculovirus polyhedron (P_{Ph}) promoter. In other embodiment, the second baculovirus genome includes a heterologous nucleic acid encoding the cap proteins under the transcriptional control of the p10 baculovirus promoter. Other modifications of the wild-type AAV sequences for proper expression in insect cells and/or to increase yield of VP and virion or to alter tropism or reduce antigenicity of the virion are also known in the art. By using helper baculoviral construct encoding the rep ORF (open reading frame) of an AAV serotype and cap ORF of a different serotype AAV, it is feasible packaging a vector flanked by ITRs of a given AAV serotype into virions assembled from structural capsid proteins of a different serotype. It is also possible by this same procedure to package mosaic, chimeric or targeted vectors.

Virus-glycan interactions are critical determinants of host cell invasion. In a particular embodiment, the AAV viral particle comprises capsid proteins comprising one or more amino acids substitutions, wherein the substitutions introduce a new glycan binding site into the AAV capsid protein. In a more particular embodiment, the amino acid substitutions are in amino acid 266, amino acids 463-475 and amino acids 499-502 in AAV2 or the corresponding amino acid positions in AAV1, AAV3, AAV4, AAV5, AAV6, AAV7, AAV 8, AAV9, AAV10 or any other AAV serotype, also included Anc80 and Anc80L65.

The introduced new glycan binding site can be a hexose binding site [e.g. a galactose (Gal), a mannose (Man), a glucose (Glu) or a fucose (fuc) binding site]; a sialic acid (Sia) binding site [e.g. a Sia residue such as is N-acetylneuraminic acid (NeuSAc) or N-Glycolylneuraminic acid (NeuSGc)]; or a disaccharide binding site, wherein the disaccharide is a sialic acid linked to galactose, for instance in the form of Sia(alpha2,3)Gal or Sia(alpha2,6)Gal. Detailed guidance to introduce a new binding site from an AAV serotype into a capsid protein of an AAV of another serotype is given on international patent publication WO2014144229 and in Shen et al. (J. Biol. Chem. 2013; 288(40):28814-28823). In a particular embodiment, the Gal binding site from AAV9 is introduced into the AAV2 VP3 backbone resulting in a dual glycan-binding AAV strain which is able to use both HS and Gal receptors for cell entry. Preferably, said dual glycan-binding AAV strain is AAV2G9. Shen et al. generated AAV2G9 by substituting amino acid residues directly involved and immediately flanking the Gal recognition site on the AAV9 VP3 capsid protein subunit onto corresponding residues on the AAV2 VP3 subunit coding region (AAV2 VP3 numbering Q464V, A467P, D469N, I470M, R471A, D472V, S474G, Y500F, and S501A).

In another embodiment, the viral particle for use according to the present disclosure may be an adenoviral particle, such as an Ad5 viral particle, which would incorporate the CYP27A1 expression cassette in the context of the appropriate vector genome. As it is the case for AAV viral particle, capsid proteins of Ad viral particles can also be engineered to modify their tropism and cellular targeting properties, alternative adenoviral serotypes can also be employed.

A Process for Producing Viral Particles

Production of viral particles carrying the expression viral vector as disclosed above can be performed by means of conventional methods and protocols, which are selected taking into account the structural features chosen for the actual embodiment of expression vector and viral particle of the vector to be produced.

Briefly, viral particles can be produced in a host cell, more particularly in specific virus-producing cell (packaging cell), which is transfected with the nucleic acid construct or expression vector to be packaged, in the presence of a helper vector or virus or other DNA construct(s).

The term “packaging cells” as used herein, refers to a cell or cell line which may be transfected with a nucleic acid construct or expression vector of the disclosure and provides in trans all the missing functions which are required for the complete replication and packaging of a viral vector. Typically, the packaging cells express in a constitutive or inducible manner one or more of said missing viral functions. Said packaging cells can be adherent or suspension cells.

These packaging cells can be either producer cell lines expressing stably helper function for AAV production or cell lines transiently expressing part or totality of helper functions.

For example, said packaging cells may be eukaryotic cells such as mammalian cells, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HEK293T cells (ATCC CRL-3216), HeLa cells (ATCC CCL2) and fetal rhesus lung cells (ATCC CL-160). Examples of non-human primate cells are Vero cells (ATCC CCL81), COS-1 cells (ATCC CRL-1650) or COS-7 cells (ATCC CRL-1651). Examples of dog cells are MDCK cells (ATCC CCL-34). Examples of rodent cells are hamster cells, such as BHK21-F, HKCC cells, or CHO cells.

As an alternative to mammalian sources, the packaging cells for producing the viral particles may be derived from avian sources such as chicken, duck, goose, quail or pheasant. Examples of avian cell lines include avian embryonic stem cells (WO01/85938 and WO03/076601), immortalized duck retina cells (WO2005/042728), and avian embryonic stem cell derived cells, including chicken cells (WO2006/108846) or duck cells, such as EB66 cell line (WO2008/129058 & WO2008/142124).

In another embodiment, the cells can be any cells permissive for baculovirus infection and replication packaging cells. In a particular embodiment, said cells are insect cells, such as SF9 cells (ATCC CRL-1711), Sf21 cells (IPLB-Sf21), MG1 cells (BTI-TN-MG1) or High Five™ cells (BTI-TN-5B1-4).

Accordingly, in a particular embodiment, the packaging cell comprises:
a nucleic acid construct or expression vector comprising a transgene encoding sterol 27-hydroxylase according to the disclosure (e.g., the AAV expression vector according to the disclosure),
a nucleic acid construct, for example a plasmid, encoding AAV rep and/or cap genes which does not carry the ITR sequences; and/or
a nucleic acid construct, for example a plasmid or virus, comprising viral helper genes.

Typically, a process of producing viral particles comprises the following steps:

a) culturing a packaging cell comprising a nucleic acid construct or expression vector as described above in a culture medium; and

b) harvesting the viral particles from the cell culture supernatant and/or inside the cells.

Conventional methods can be used to produce AAV viral particles which consist on transient cell co-transfection of cell lines with nucleic acid construct or expression vector (e.g. a plasmid) carrying the transgene of the disclosure; a nucleic acid construct (e.g., an AAV helper plasmid) that encodes rep and cap genes, but does not carry ITR sequences; and with a third nucleic acid construct (e.g., a plasmid) providing the adenoviral functions necessary for AAV replication. Viral genes necessary for AAV replication are referred herein as viral helper genes. Typically, said genes necessary for AAV replication are adenoviral helper genes, such as E1A, E1B, E2a, E4, or VA RNAs. Preferably, the adenoviral helper genes are of the Ad5 or Ad2 serotype.

Large-scale production of AAV particles according to the disclosure can also be carried out for example by infection of insect cells with a combination of recombinant baculoviruses (Urabe et al. Hum. Gene Ther. 2002; 13: 1935-1943). SF9 cells are co-infected with two or three baculovirus vectors respectively expressing AAV rep, AAV cap and the AAV vector to be packaged. The recombinant baculovirus vectors will provide the viral helper gene functions required for virus replication and/or packaging. Smith et al 2009 (Molecular Therapy, vol. 17, no. 11, pp 1888-1896) further describes a dual baculovirus expression system for large-scale production of AAV particles in insect cells.

Suitable culture media will be known to a person skilled in the art. The ingredients that compose such media may vary depending on the type of cell to be cultured. In addition to nutrient composition, osmolarity and pH are considered important parameters of culture media. The cell growth medium comprises a number of ingredients well known by the person skilled in the art, including amino acids, vitamins, organic and inorganic salts, sources of carbohydrate, lipids, trace elements (CuS04, FeS04, Fe(N03)3, ZnS04 . . . ), each ingredient being present in an amount which supports the cultivation of a cell in vitro (i.e., survival and growth of cells). Ingredients may also include different auxiliary substances, such as buffer substances (like sodium bicarbonate, Hepes, Tris . . . ), oxidation stabilizers, stabilizers to counteract mechanical stress, protease inhibitors, animal growth factors, plant hydrolyzates, anti-clumping agents, anti-foaming agents. Characteristics and compositions of the cell growth media vary depending on the particular cellular requirements. Examples of commercially available cell growth media are: MEM (Minimum Essential Medium), BME (Basal Medium Eagle) DMEM (Dulbecco's modified Eagle's Medium), Iscoves DMEM (Iscove's modification of Dulbecco's Medium), GMEM, RPMI 1640, Leibovitz L-15, McCoy's, Medium 199, Ham (Ham's Media) F10 and derivatives, Ham F12, DMEM/F12, etc.

Following viral particles production, viral particle can be purified from the host cell using a variety of conventional purification methods, such as column chromatography, CsCl gradients, and the like. For example, a plurality of column −36-purification steps can be used, such as purification over an anion exchange column, an affinity column and/or a cation exchange column. Further, if infection is employed to express the accessory functions, residual helper virus can be inactivated, using known methods.

The resulting viral particle comprising a transgene encoding sterol 27-hydroxylase according to the disclosure can be used for gene therapy using the techniques described below.

Further guidance for the construction and production of viral vectors for use according to the disclosure can be found in Viral Vectors for Gene Therapy, Methods and Protocols. Series: Methods in Molecular Biology, Vol. 737. Merten and Al-Rubeai (Eds.); 2011 Humana Press (Springer); Gene Therapy. M. Giacca. 2010 Springer-Verlag; Heilbronn R. and Weger S. Viral Vectors for Gene Transfer: Current Status of Gene Therapeutics. In: Drug Delivery, Handbook of Experimental Pharmacology 197; M. Schafer-Korting (Ed.). 2010 Springer-Verlag; pp. 143-170; Adeno-Associated Virus: Methods and Protocols. R. O. Snyder and P. Moulllier (Eds). 2011 Humana Press (Springer); Bunning H. et al. Recent developments in adeno-associated virus technology. J. Gene Med. 2008; 10:717-733; Adenovirus: Methods and Protocols. M. Chillón and A. Bosch (Eds.); Third Edition. 2014 Humana Press (Springer)

Host Cells

In another aspect, the disclosure relates to a host cell comprising a nucleic acid construct or an expression vector of the disclosure. More particularly, host cell according to the disclosure is a specific virus-producing cell, also named packaging cell which is transfected with the nucleic acid construct or expression vector according to the disclosure, in the presence of a helper vector or virus or other DNA constructs and provides in trans all the missing functions which are required for the complete replication and packaging of a viral particle. Said packaging cells can be adherent or suspension cells

For example, said packaging cells may be eukaryotic cells such as mammalian cells, including simian, human, dog and rodent cells. Examples of human cells are PER.C6 cells (WO01/38362), MRC-5 (ATCC CCL-171), WI-38 (ATCC CCL-75), HEK-293 cells (ATCC CRL-1573), HEK293T cells (ATCC CRL-3216), HeLa cells (ATCC CCL2) and fetal rhesus lung cells (ATCC CL-160). Examples of non-human primate cells are Vero cells (ATCC CCL81), COS-1 cells (ATCC CRL-1650) or COS-7 cells (ATCC CRL-1651). Examples of dog cells are MDCK cells (ATCC CCL-34). Examples of rodent cells are hamster cells, such as BHK21-F, HKCC cells, or CHO cells.

As an alternative to mammalian sources, the packaging cells for producing the viral particles may be derived from avian sources such as chicken, duck, goose, quail or pheasant. Examples of avian cell lines include avian embryonic stem cells (WO01/85938 and WO03/076601), immortalized duck retina cells (WO2005/042728), and avian embryonic stem cell derived cells, including chicken cells (WO2006/108846) or duck cells, such as EB66 cell line (WO2008/129058 & WO2008/142124).

In another embodiment, the cells can be any cells permissive for baculovirus infection and replication packaging cells. In a particular embodiment, said cells are insect cells, such as SF9 cells (ATCC CRL-1711), Sf21 cells (IPLB-Sf21), MG1 cells (BTI-TN-MG1) or High Five™ cells (BTI-TN-5B1-4).

Accordingly, in a particular embodiment, the host cell comprises:
a nucleic acid construct or expression vector comprising a transgene encoding sterol 27-hydroxylase according to the disclosure (e.g., the AAV expression vector according to the disclosure),
a nucleic acid construct, for example a plasmid, encoding AAV rep and/or cap genes which does not carry the ITR sequences; and/or
a nucleic acid construct, for example a plasmid or virus, comprising viral helper genes.

In another aspect, the disclosure relates to a host cell transduced with a viral particle of the disclosure and the term “host cell” as used herein refers to any cell line that is susceptible to infection by a virus of interest, and amenable to culture in vitro.

The host cell of the disclosure may be used for ex vivo gene therapy purposes. In such embodiments, the cells are transduced with the viral particle of the disclosure and subsequently transplanted to the patient or subject. Transplanted cells can have an autologous, allogenic or heterologous origin. For clinical use, cell isolation will generally be carried out under Good Manufacturing Practices (GMP) conditions. Before transplantation, cell quality and absence of microbial or other contaminants is typically checked and liver preconditioning, such as with radiation and/or an immunosuppressive treatment, may be carried out. Furthermore, the host cells may be transplanted together with growth factors to stimulate cell proliferation and/or differentiation, such as Hepatocyte Growth Factor (HGF).

In a particular embodiment, the host cell is used for ex vivo gene therapy into the liver. Preferably, said cells are eukaryotic cells such as mammalian cells, these include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like. A person skilled in the art will choose the more appropriate cells according to the patient or subject to be transplanted.

Said host cell may be a cell with self-renewal and pluripotency properties, such as stem cells or induced pluripotent stem cells. Stem cells are preferably mesenchymal stem cells. Mesenchymal stem cells (MSCs) are capable of differentiating into at least one of an osteoblast, a chondrocyte, an adipocyte, or a myocyte and may be isolated from any type of tissue. Generally, MSCs will be isolated from bone marrow, adipose tissue, umbilical cord, or peripheral blood. Methods for obtaining thereof are well known to a person skilled in the art. Induced pluripotent stem cells (also known as iPS cells or iPSCs) are a type of pluripotent stem cell that can be generated directly from adult cells. Yamanaka et al. induced iPS cells by transferring the Oct3/4, Sox2, Klf4 and c-Myc genes into mouse and human fibroblasts, and forcing the cells to express the genes (WO 2007/069666). Thomson et al. subsequently produced human iPS cells using Nanog and Lin28 in place of Klf4 and c-Myc (WO 2008/118820).

Said host cells may also be hepatocytes. Hepatocyte transplantation procedures, including cell isolation and subsequent transplantation into a human or mice recipient is described for instance in Filippi and Dhawan, Ann NY Acad Sci. 2014, 1315 50-55; Yoshida et al., Gastroenterology 1996, 111: 1654-1660; Irani et al. Molecular Therapy 2001, 3:3, 302-309; and Vogel et al. J Inherit Metab Dis 2014, 37:165-176. A method for ex vivo transduction of a viral particle into hepatocytes is described for instance in Merle et al., Scandinavian Journal of Gastroenterology 2006, 41:8, 974-982.

Pharmaceutical Compositions

Another aspect of the present disclosure relates to a pharmaceutical composition comprising a nucleic acid construct, expression vector, viral particle or host cell of the disclosure in combination with one or more pharmaceutical acceptable excipient, diluent or carrier.

As used herein, the term “pharmaceutically acceptable” means approved by a regulatory agency or recognized pharmacopeia such as European Pharmacopeia, for use in animals and/or humans. The term “excipient” refers to a diluent, adjuvant, carrier, or vehicle with which the therapeutic agent is administered.

Any suitable pharmaceutically acceptable carrier, diluent or excipient can be used in the preparation of a pharmaceutical composition (See e.g., Remington: The Science and Practice of Pharmacy, Alfonso R. Gennaro (Editor) Mack Publishing Company, April 1997).

Pharmaceutical compositions are typically sterile and stable under the conditions of manufacture and storage. Pharmaceutical compositions may be formulated as solutions (e.g. saline, dextrose solution, or buffered solution, or other pharmaceutically acceptable sterile fluids), microemulsions, liposomes, or other ordered structure suitable to accommodate a high product concentration (e.g. microparticles or nanoparticles). The carrier may be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

Prolonged absorption of the injectable compositions can be brought about by including in the composition an agent which delays absorption, for example, monostearate salts and gelatin. The product of the disclosure may be administered in a controlled release formulation, for example in a composition which includes a slow release polymer or other carriers that protect the product against rapid release, including implants and microencapsulated delivery systems. Biodegradable and biocompatible polymers may for example be used, such as ethylene vinyl acetate, polyanhydrides, polyglycolic acid, collagen, polyorthoesters, polylactic acid and polylactic/polyglycolic copolymers (PLG). Preferably, said pharmaceutical composition is formulated as a solution, more preferably as an optionally buffered saline solution. Supplementary active compounds can also be incorporated into the pharmaceutical compositions of the disclosure. Guidance on co-administration of additional therapeutics can for example be found in the Compendium of Pharmaceutical and Specialties (CPS) of the Canadian Pharmacists Association.

In one embodiment, the pharmaceutical composition is a parenteral pharmaceutical composition, including a composition suitable for intravenous, intraarterial, subcutaneous, intraperitoneal or intramuscular administration. These pharmaceutical compositions are exemplary only and do not limit the pharmaceutical compositions suitable for other parenteral and non-parenteral administration routes. The pharmaceutical compositions described herein can be packaged in single unit dosage or in multidosage forms.

Therapeutic Uses

In a further aspect, the disclosure relates to a nucleic acid construct, expression vector, viral particle, host cell or pharmaceutical composition of the disclosure for use as a medicament in a subject in need thereof.

The term “subject” or “patient” as used herein, refers to mammals. Mammalian species that can benefit from the disclosed methods of treatment include, but are not limited to, humans, non-human primates such as apes, chimpanzees, monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

In an additional aspect, the disclosure relates to a nucleic acid construct, expression vector, viral particle, host cell or pharmaceutical composition of the disclosure for use in the treatment of Cerebrotendinous Xanthomatosis in a subject in need thereof.

Cerebrotendinous Xanthomatosis (CTX) is an anomaly of bile acid synthesis characterized by neonatal cholestasis, childhood onset cataract, adolescent to young adult-onset tendon xanthomata and brain xanthomata with adult-onset neurologic dysfunction. CTX is caused by mutations in the sterol 27-hydroxylase gene. Sterol 27-hydroxylase catalyzes the first step in the oxidation of the side-chain of sterol intermediates in the bile acid synthesis (BAS) pathway. Defective enzymatic function disrupts bile acid synthesis leading to cholesterol and cholestanol deposits, which result in a degenerative process.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. According to the present disclosure, examples of symptoms associated with CTX may be neonatal cholestasis or chronic diarrhea from infancy, cataract, cholestasis and liver dysfunction, xanthomata in the achilles and other tendons (elbow, hand, patella, neck), intellectual impairment from infancy, adult-onset progressive neurologic dysfunction which includes dementia, psychiatric disturbances, pyramidal and/or cerebellar signs, seizures, and neuropathy. In other embodiments, this term refers to minimizing the spread or worsening of the disease resulting from the administration of one or more therapeutic agents to a subject with such a disease.

In a related aspect, the disclosure pertains to the use of a nucleic acid construct, expression vector, viral particle, host cell or pharmaceutical composition of the disclosure in the preparation of a medicament for use in the treatment of a liver disease, preferably for use in the treatment of CTX.

In a further aspect, the disclosure relates to a method of treating and/or preventing a liver disease, preferably CTX, in a subject in need thereof that comprises administering to the subject a therapeutically effective amount of a nucleic acid construct, expression vector, viral particle, host cell or pharmaceutical composition of the disclosure.

In the context of the disclosure, an “effective amount” means a therapeutically effective amount.

As used herein a “therapeutically effective amount” refers to an amount effective, at dosages and for periods of time necessary to achieve the desired therapeutic result, such as amelioration or restoration of bile salts synthesis, normalization of bile acid precursors and by-products in the blood and other organs, reduction of cholesterol and cholestanol deposits and amelioration or stabilization of neurological manifestations. The therapeutically effective amount of the product of the disclosure, or pharmaceutical composition that comprises it may vary according to factors such as the disease state, age, sex, and weight of the individual, and the ability of the product or pharmaceutical composition to elicit a desired response in the individual. Dosage regimens may be adjusted to provide the optimum therapeutic response. A therapeutically effective amount is also typically one in which any toxic or detrimental effect of the product or pharmaceutical composition is outweighed by the therapeutically beneficial effects.

The treatment with a product of the disclosure may alleviate, ameliorate, or reduce the severity of one or more symptoms of CTX. For example, treatment may increase and/or restore bile salts synthesis; decrease the amount of cholesterol and cholestanol deposits in different organs, and as a consequence may alleviate, ameliorate, or reduce the severity of the disease.

The product of the disclosure will be typically included in a pharmaceutical composition or medicament, optionally in combination with a pharmaceutical carrier, diluent and/or adjuvant. Such composition or medicinal product comprises the product of the disclosure in an effective amount, sufficient to provide a desired therapeutic effect, and a pharmaceutically acceptable carrier or excipient.

In one embodiment the nucleic acid construct, expression vector, viral particle, host cell or pharmaceutical composition for its therapeutic use is administered to the subject or patient by a parenteral route, in particularly by intravenous, intraarterial, subcutaneous, intraperitoneal, or intramuscular route.

In one embodiment, the nucleic acid construct, expression vector, viral particle, host cell or pharmaceutical composition for its therapeutic use is administered by interstitial route, i.e. by injection to or into the interstices of a tissue. The tissue target may be specific, for example the liver tissue, or it may be a combination of several tissues, for example the muscle and liver tissues. Exemplary tissue targets may include liver, skeletal muscle, heart muscle, adipose deposits, kidney, lung, vascular endothelium, epithelial and/or hematopoietic cells. In a preferred embodiment, it is administered by intrahepatic injection, i.e. injection into the interstitial space of hepatic tissue.

The amount of product of the disclosure that is administered to the subject or patient may vary depending on the particular circumstances of the individual subject or patient including, age, sex, and weight of the individual; the nature and stage of the disease, the aggressiveness of the disease; the route of administration; and/or concomitant medication that has been prescribed to the subject or patient. Dosage regimens may be adjusted to provide the optimum therapeutic response.

For any particular subject, specific dosage regimens may be adjusted over time according to the individual needs and the professional judgment of the person administering or supervising the administration of the compositions. Dosage ranges set forth herein are exemplary only and do not limit the dosage ranges that may be selected by medical practitioners.

In one embodiment, an AAV viral particle according to the disclosure can be administered to the subject or patient for the treatment of CTX disease in an amount or dose comprised within a range of 1×10⁸ to 1×10¹⁴ vg/kg (vg: viral genomes; kg: subject's or patient's body weight). In a more particular embodiment, the AAV viral particle is administered in an amount comprised within a range of 1×10¹¹ to 1×10¹⁴ vg/kg. In a more particular embodiment, the AAV viral particle is administered at a dosage of at least 1×10¹² vg/kg, preferably 5×10¹² vg/kg, more preferably 1×10¹³ vg/kg, and more preferably 5×10¹³ vg/kg.

Kit

In another aspect, the disclosure further relates to a kit comprising a nucleic acid construct, expression vector, host cell, viral particle or pharmaceutical composition as described above in one or more containers. The kit may include instructions or packaging materials that describe how to administer the nucleic acid construct, expression vector, viral particle, host cell or pharmaceutical compositions contained within the kit to a patient. Containers of the kit can be of any suitable material, e.g., glass, plastic, metal, etc., and of any suitable size, shape, or configuration. In certain embodiments, the kits may include one or more ampoules or syringes that contain the products of the disclosure in a suitable liquid or solution form.

The following examples are provided by way of illustration, and they are not intended to be limiting of the present disclosure. Furthermore, the present disclosure covers all possible combinations of particular and preferred embodiments described herein.

### EXAMPLES

Materials and Methods

Cell Culture

HuH-7 (JCRB0403), HepG2 (ATCC HB-8065), Hep3B (ATCC HB-8064), 293T (ATCC CRL-3216), Hepa 1-6 (ATCC CRL-1830), AML12 (ATCC CRL-2254) cell lines were maintained in Dulbecco's Modified Eagle Medium (DMEM)-high glucose (Sigma-Aldrich, St. Louis, Mo.) supplemented with 10% foetal bovine serum (FBS, Invitrogen™, Thermo Fisher Scientific, Carlsbad, Calif.), 100 U/mL penicillin, 100 μg/mL streptomycin, 2 mM L-glutamine and 1% non-essential amino acids (Gibco™, Thermo Fisher Scientific, Waltham, Mass.). The AML12 cell line (ATCC CRL-2254) was maintained in DMEM/F12 Medium (Gibco™, Thermo Fisher Scientific), supplemented with 10% FBS, 0.005 mg/ml insulin, 0.005 mg/ml transferrin, 5 ng/ml selenium (Gibco™, Thermo Fisher Scientific), 40 ng/ml dexamethasone, 100 U/mL penicillin and 100 μg/mL streptomycin. All cells were maintained at 37° C. in a 5% CO₂ atmosphere.

Luciferase Reporter Plasmids

The pGL3-Basic plasmid (Promega, Madison, Wis.) is a promoter-less construct used to determine the background luciferase expression. The pCMV-Luc and pEalbPa1AT-Luc plasmids have been already described (Kramer, M. G et al. (2003). Mol. Ther. 7, 375-385). The EalbPa1AT-Luc promoter (hereinafter referred to as EAAT) is a liver-specific, hybrid regulatory sequence consisting of the mouse albumin enhancer linked to the human α1-antitrypsin promoter. The pC27P-Luc plasmid contains a regulatory sequence comprising 2024 bp upstream of the human CYP27A1 translation initiation site (Araya Z, et al. Biochem. J. 2003; 372:529-534; Chen W, et al. Gene. 2003; 313:71-82) synthetized by GenScript (Piscataway, N.J.) and introduced into the MluI-NheI sites of pGL3-Basic.

Transfection and Luciferase Assays

All cell lines were seeded in 24-well plates at a density of 10⁵ cells per well and, 24 h later, they were transfected with Lipofectamine 2000 (Invitrogen™, Thermo Fisher Scientific) using 1 μg of plasmid DNA and 2 μg of Lipofectamine per well. Five hours later, medium was refreshed and cells were maintained for 48 h before addition of the Passive Lysis Buffer 5× (Promega, Madison, Wis.). Luciferase activity was measured with the Luciferase Reporter Assay System (Promega) in a Luminat KB 9507 Luminometer (Berthold Technologies, Bad Wildbad, Germany). Data were normalized by protein content in each sample (in μg), determined by the Bradford assay (Biorad, Hercules, Calif.). Promoter activity was represented as percentage of luciferase activity, using the CMV promoter as a reference.

AAV Vectors

AAV-EAAT-CYP27A1 and AAV-C27P-CYP27A1 are AAV8 vectors containing the CYP27A1 cDNA under the control of the EAAT or CYP27A1 promoters, respectively. For the construction of the AAV-EAAT-CYP27A1 genome (pAAV-EAAT-CYP27A1 plasmid), the CYP27A1 coding sequence (NCBI ID. CCDS2423.1) was synthetized by GenScript Biotech (Leiden, Netherlands). This DNA fragment was introduced using NheI and XbaI sites into a plasmid containing the EAAT promoter and a poly-adenylation site, flanked by inverted terminal repeats (ITRs) from AAV2. The pAAV-EAAT-Luc plasmid contains the Firefly luciferase under the control of the EAAT promoter. For construction of the pAAV-C27P-CYP27A1 plasmid, the CYP27A1 promoter was excised from the pC27P-Luc plasmid using MluI and NheI sites, and introduced into the same sites of pAAV-EAAT-CYP27A1, thus replacing the EAAT promoter. For viral particle (VP) production, the plasmids were transfected together with the pDP8-ape helper plasmid (Plasmid Factory, Bielefeld, Germany) in 293-T cells, using polyethyleneimine (Polysciences, Warrington, Pa.). Three days later, culture media and cells were separated by centrifugation. VPs were extracted from the cell pellet by addition of lysis buffer (50 mM Tris-Cl, 150 mM NaCl, 2 mM MgCl2, 0.1% Triton X-100) and 3 cycles of freezing and thawing (−80° C.). VPs in the culture media were precipitated using polyethylene glycol solution (PEG8000, 8% v/v final concentration, Sigma-Aldrich) for 48-72 h at 4° C. and further centrifugation at 1378×g for 15 min. The pellet was resuspended in lysis buffer and kept at −80° C. VPs obtained from culture medium and cell lysates were purified by ultracentrifugation at 350,000 g during 2.5 h in a 15-57% iodioxanol gradient. Finally, the purified viruses were concentrated using Amicon Ultra Centrifugal Filters-Ultracel 100K (Millipore, Burlington, Mass.). Quantification of AAV vectors was performed by quantitative PCR (qPCR). To this end, VPs were treated with DNAse and then viral genomes were extracted using the High Pure Viral NucleicAcid Kit (Roche, Indianapolis, Ind.). Primers are listed in Table 1.

TABLE 1
List of primers

| | | | SEQ ID |
|---|---|---|---|
| Gene | Primer | Sequence | NO |
| Human | Forward | 5′ TGTGCTTAAGGAGACTCTGCG 3′ | 10 |
| CYP27A1 | Reverse | 5′ ATAGTGGCAGAACACAAACTGG 3′ | 11 |
| Mouse | Forward | 5′ ACAAGGCTATGTGCTGCACTTG 3′ | 12 |
| Cyp27a1 | Reverse | 5′ TGATCCATGTGGTCTCTTATTG 3′ | |
| exons 1/2 | | | 13 |
| Mouse | Forward | 5′ ATGGCTGAGGAAGAAAGAGG 3′ | 14 |
| Cyp27a1 | Reverse | 5′ ACACAGTCTTTACTTCTCCCATC 3′ | |
| exons 8/9 | | | 15 |
| Mouse | Forward | 5′ GAAGCAATGAAAGCAGCCTC 3′ | 16 |
| Cyp7a1 | Reverse | 5′ GTAAATGGCATTCCCTCCAG 3′ | 17 |
| Mouse | Forward | 5′ TGAATATGAAACTTGCTCTCACTAAAA 3′ | 18 |
| Cyp3a11 | Reverse | 5′ CCTTGTCTGCTTAATTTCAGAGGT 3′ | 19 |
| Mouse 36b4 | Forward | 5′ AACAATCTCCCCCTTCTCCTT 3′ | 20 |
| | Reverse | 5′ GAAGGCCTTGACCTTTTCAG 3′ | 21 |

Animals and Husbandry

A mouse strain with truncation of Cyp27a1 exon 8 was obtained from The Jackson Laboratory (Bar Harbor, Me.) (B6.129-Cyp27a1^{tm1Et}/J, Ref. 009106) (Rosen H, et al. J. Biol. Chem. 1998; 273:14805-14812). Mice homozygous for this mutation (referred hereinafter to as Cyp27a1^{-/-} or CTX mice) were maintained in a C57BL6/J background by crossing heterozygous individuals. The offspring was genotyped after weaning as indicated by the repository.

Animals were group-housed, up to 6 animals per cage (male or female), provided with food and water ad libitum and maintained with a 12 h light-dark cycle. The average age for initiation of studies was 7 weeks. AAV vectors were administered intravenously by retro-orbital injection in a final volume of 150 μl saline solution. Chenodeoxycholic (CDCA) (Sigma-Aldrich, Ref: C9377-25G) was supplemented in standard chow at 0.1, 0.5 and 1 g CDCA/100 g of chow (0.1, 0.5 and 1.0% CDCA diets, respectively). Blood was collected by submandibular venous puncture using 1.3 ml EDTA tubes (Sarstedt, Nimbrecht, Germany) except for end-time points/terminal procedures, in which cardiac puncture was performed in anesthetized mice. Once animals were euthanized, liver samples were collected for histological and gene expression analyses.

All procedures were performed and approved by the ethical Committee of the Universidad de Navarra, according to the Spanish Royal Decree 53/2013.

Hydrodynamic Injection and Bioluminescence Imaging

For in vivo liver transfection, 20 μg of reporter plasmids diluted in 1.8 ml saline were injected as a bolus through the lateral tail vein (Kramer M G, et al. Mol. Ther. 2003; 7:375-385). Luciferase activity was determined 48 h later by bioluminescence imaging (BLI). To this end, mice were briefly anesthetized with an injection of a ketamine/xylazine mixture (80:10 mg/kg, i.p.). The substrate D-luciferin (REGIS Technologies, Morton Grove, Ill.) was administered intraperitoneally (100 μl of a 30 μg/μl solution in PBS). Light emission was detected 5, 20 and 30 min later using a PhotonImager™ Optima apparatus (BioSpace Lab, Nesles-la-Vallée, France). Data were analyzed using the M3Vision software (BioSpace Lab), representing the maximal value obtained for each animal.

Biochemical Analyses in Plasma

Blood was centrifuged at 10.000 g for 5 min at room temperature. Plasma was treated with 20 μM butylhydroxytoluene (Sigma) in a N2 atmosphere to protect from oxidation before storage at −80° C. in opaque tubes. Sterol extractions were performed using 100 μl of plasma for quantification of cholestanol and 7αC4 concentrations by HPLC-MS/MS as previously described (Chen W, Chiang J Y L. Gene. 2003; 313:71-82). Bile acid (BA) profiling in serum was carried out after acetonitrile precipitation/extraction (Leníček M, et al. J. Chromatogr. B Anal. Technol. Biomed. Life Sci. 2016; 1033-1034:317-320), using an adaptation (Nytofte N S, et al. J. Med. Genet. 2011; 48:219-225). of a previously described method for BA measurement by HPLC-MS/MS (Ye L, et al. J. Chromatogr. B Anal. Technol. Biomed. Life Sci. 2007; 860:10-17) on a 6420 Triple Quad LC/MS device (Agilent Technologies, Santa Clara, Calif.) (Monte M J, et al. J. Hepatol. 2002; 36:534-542). Alanine aminotransferase (ALT) was quantified in 40 μl plasma samples using a Cobas C311 automated chemistry analyser (Roche Diagnostics, Basel, Switzerland).

Quantitative PCR

RNA was extracted from frozen liver samples using the Maxwell® 16 LEV simplyRNA Cells Kit (Promega) following manufacture's recommendations. Two μg of RNA, treated with DNase I, were retro-transcribed using M-MLV retro-transcriptase (Invitrogen™) and random primers (Life Technologies, Thermo). cDNA was amplified and relative gene expression was determined by quantitative polymerase chain reaction (qPCR) using iQ™ SYBR® Green Supermix reagent (Bio-Rad), in CFX96 Touch™ Real-Time PCR Detection System (Bio-Rad). Table 1 contains the sequence of primers specific for the transgene (CYP27A1) and the mouse genes Cyp7a1, Cyp3a11, Cyp27a1 (exons 1/2), Cyp27a1 (exons 8/9) and 36b4 (used as a housekeeping gene).

ΔCt values using 36b4 mRNA levels as reference gene were corrected with the efficiency of amplification of each pair of primers and multiplied by 1.000 to facilitate graphical representation.

Western Blot

Total proteins were isolated from liver samples using RIPA buffer (NaCl 200 mM, HEPES 100 mM, Glicerol 10%, NaF 200 mM, Na4P2O7 2 mM, EDTA 5 mM, EGTA 1 mM, DTT 2 mM (Invitrogen™), PMSF 0.5 mM, Na3VO41 mM and Complete™ Protease Inhibitor Cocktail (Roche)). Twenty μg of total protein extracts were boiled for 1 min and electrophoresed on a 10% polyacrylamide gel. Transfer to nitrocellulose membranes was performed at 340 mA current intensity for 3 hours at 4° C. Next, membranes were incubated for 1 h at room temperature in blocking solution (5% bovine serum albumin in TBS-Tween) followed by overnight incubation at 4° C. with primary antibodies diluted in 1% BSA, 0.05% Tween-20 and 0.5% sodium azide in TBS. Primary antibodies are anti-CYP27a1 (Abcam, Cambridge, UK, Cat #EPR7529, Cat #ab126785, 1:1.000) 1) and anti-GAPDH (Cell Signaling Technology, Danvers, Mass., 1:5.000). After washing with 0.1% Tween-20 in TBS, membranes were incubated for 1 h with anti-rabbit IgG HRP conjugate secondary antibody (GE Healthcare, Chicago, Ill., Cat #NA934V, 1:10.000). Images were acquired with a Chemidoc system (Bio-Rad), and Image Lab™ software (Bio-Rad) was used for quantification.

Immunohistochemistry

For detection of CYP27A1 in hepatocytes, 3 m thick sections cut from liver samples fixed in 4% paraformaldehyde and embedded in paraffin were deparaffinized with xylene, hydrated with decreasing concentrations of ethanol, and incubated with 3% hydrogen peroxide to block endogenous peroxidase. Antigen retrieval was performed by heating in 10 mM Citrate buffer pH 6 or 10 mM Tris-EDTA buffer for 20 minutes before incubation with antibody for CYP27A1 (Abcam, Cambridge, UK, Cat #ab126785, 1:250) and β-Catenin (Cell Signaling Technology, Danvers, Mass., Cat #8480, 1:250) respectively. HRP-conjugated Envision secondary antibody (K4003, DAKO, Glostrup, Denmark) followed by DAB reagent (K3468, DAKO) were applied for the detection procedure. Tissue sections were counterstained with Hematoxylin (Sigma-Aldrich) and dehydrated. Negative controls were included omitting primary antibodies. Quantification of hepatocytes overexpressing the protein was performed in 5 fields per mice (311×311 μm) using ImageJ software (NIH, Bethesda, Md.).

Statistical Analysis

The GraphPad Prism software was used for analysis. Data sets following normal distribution (D'Agostino and Pearson normality test) were compared using 1-way ANOVA with Sidak's multiple comparisons tests. Otherwise, groups were compared using Kruskal-Wallis with Dunn's post-test.

Results

### Example 1

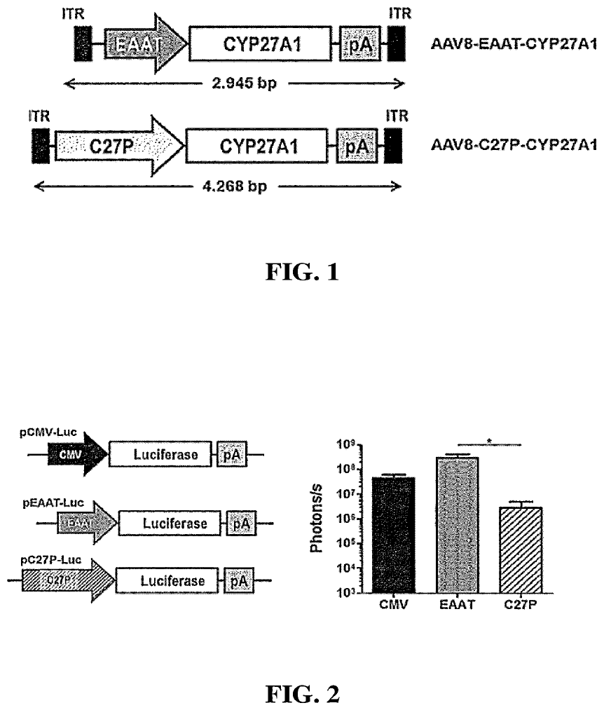

The expression cassette in the AAV8-EAAT-CYP27A1 vector contains the human CYP27A1 cDNA under the control of a hybrid promoter (hereinafter called EAAT) based on the mouse albumin enhancer and the human alpha-1-antitrypsin promoter (FIG. 1). This strong liver-specific regulatory sequence was described by Kramer et al. Mol. Ther. 7 (2003) 375-385.

The expression cassette in the AAV8-C27P-CYP27A1 vector contains the human CYP27A1 cDNA under the control of the 2 kb 5′ UTR region of the human CYP27A1 gene, where the main regulatory elements of this gene have been identified (Chen et al. Gene 313 (2003) 71-82, Araya et al. Biochem. J. 372 (2004) 71-82). This sequence is hereinafter called C27P (FIG. 1).

Evaluation of Promoter In Vivo

In order to evaluate the relative potency of both promoters, the inventors obtained reporter plasmids in which the luciferase coding sequence is controlled by these sequences (FIG. 2). The plasmids were transfected in the liver of C57BL/6 mice by hydrodynamic injection, and 48 hours later the luciferase activity was evaluated in the liver by bioluminescence imaging. To this end, 25 μg of each plasmid was dissolved in 2.5 ml of saline solution and injected during 5 seconds through the tail vein. For bioluminescence analysis (BLI), mice were anesthetized by inhalation of 2% isofluorane. The substrate D-luciferine (150 mg/kg in 100 μl volume) was injected intraperitoneally, and light emission was quantified (in photons/second) 5, 15 and 30 minutes later. The peak value was used for the representation. The result shows that the EAAT promoter achieves 100 times more activity than the C27P promoter in this animal model.

Metabolic Correction of Cyp27a k.o. Mice

Next, the inventors proceeded to the therapeutic evaluation of the AAV8-EAAT-CYP27A1 and AAV8-C27P-CYP27A1 vectors in C57BL/6 mice harboring a truncation of the Cyp27a1 gene (hereinafter called Cyp27a1 k.o.). This is the only CTX animal model available to date (Rosen et al. J. Biol. Chem. 273 (1998) 14805-14812). The vectors were administered to 6 weeks-old Cyp27a1 k.o. mice by intravenous administration using the following doses: 1.5×10¹² vg/kg and 1.5×10¹³ vg/kg for AAV8-EAAT-CYP27A1, and 5×10¹² vg/kg and 1.5×10¹³ vg/kg for AAV8-C27P-CYP27A1.

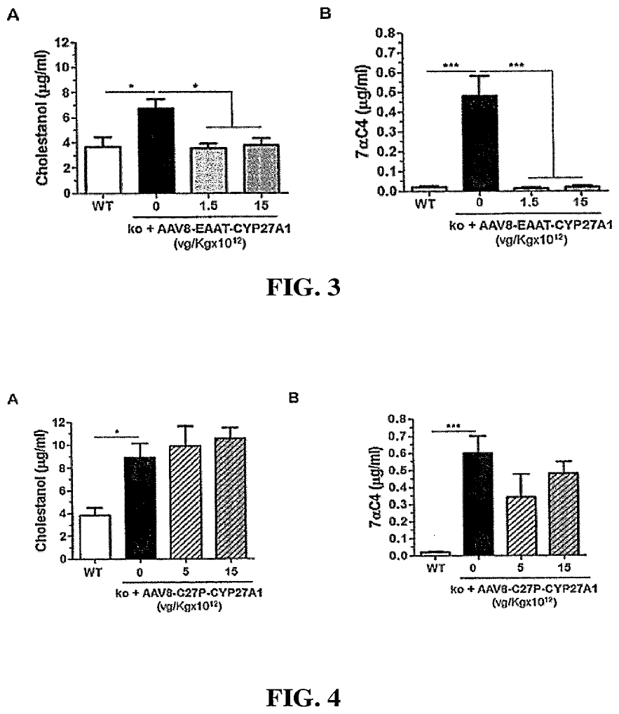

Blood was collected 15 days later for determination of cholestanol and 7aC4 in plasma by HPLC/mass spectrometry. The result demonstrates that AAV8-EAAT-CYP27A1 is able to normalize both metabolites, even at the lowest dose tested (FIG. 3), whereas AAV8-C27P-CYP27A1 has a marginal effect at the highest dose tested (FIG. 4). Of note, neither elevation of liver transaminases nor any other sign of toxicity was detected in any of the treated animals.

Expression of CYP27A1 in the Liver of Mice Treated with AAV Vectors

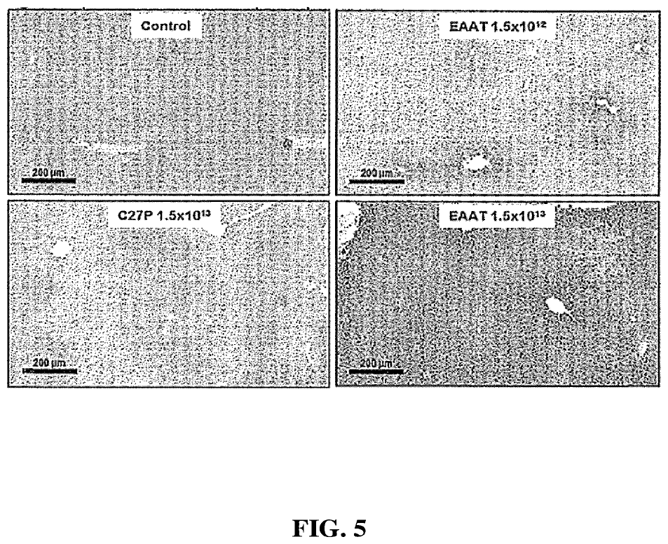

Mice were sacrificed after blood collection in order to analyze the expression of human CYP27A1 in the liver. Tissue samples were fixed in formaldehyde/ethanol and embedded in paraffin. Detection of CYP27A1 in tissue slices was carried out by immunohistochemistry using and antibody recognizing the human and mouse protein. Staining was obtained by reaction of the antibody-linked horseradish peroxidase with the substrate DAB, giving rise to a brown precipitate. Images were captured with a digital camera coupled to an optical microscope. As expected, mice treated with the low dose of AAV8-EAAT-CYP27A1 (1.5×10¹² vg/kg) present a small proportion of hepatocytes strongly labelled with the anti-CYP27A1 antibody (FIG. 5).

The transduced hepatocytes are localized in the peri-venous area, which coincides with the hepatic zone in which bile acids are physiologically synthetized. At the highest dose tested (1.5×10¹³ vg/kg), the vast majority of hepatocytes show intense production of human CYP27A I. In sharp contrast, mice treated with 1.5×10¹² vg/kg of AAV8-C27-CYP27A1 show a faint labelling, indicating lower production of the therapeutic protein.

In summary, these results indicate that high level of CYP27A1 production in a small fraction of hepatocytes can act as a sink to eliminate the excess of intermediate metabolites generated in genetic metabolic diseases such as CTX. This can be achieved with relatively low doses of GT vectors equipped with strong promoters, which increases the feasibility of this approach in the clinical practice.

### Example 2

An AAV Vector Equipped with a Liver-Specific Promoter Achieves Efficient Expression of CYP27A1 in CTX Mice

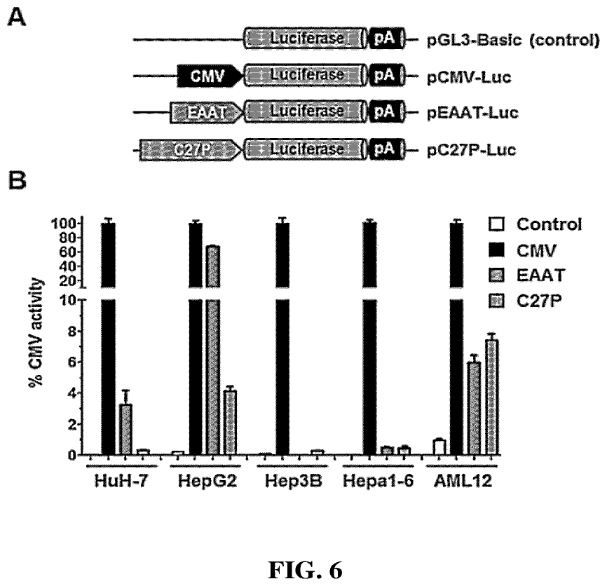

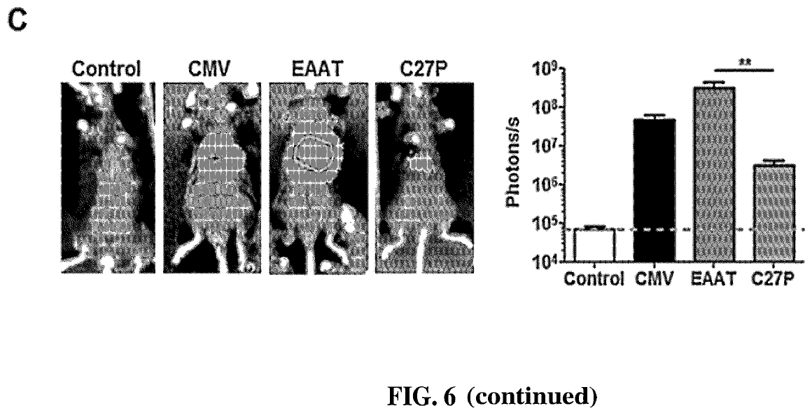

The present therapeutic approach is based on expression of CYP27A1 in the liver. For the design of the expression cassette, the inventors compared the performance of two different regulatory sequences, depicted in FIG. 6A: (i) a well-established hybrid liver-specific promoter (EAAT) (Kramer M G et al. Mol. Ther. 2003; 7:375-385), and (ii) the endogenous CYP27A1 regulatory sequence comprising 2024 bp upstream of the translation start site (Araya Z, et al. Biochem. J. 2003; 372:529-534; Chen W, Chiang J Y L. Gene. 2003; 313:71-82) (referred hereinafter as C27P). The purpose was to determine if the C27P promoter was suitable for regulation of transgene expression. Both sequences were first incorporated in luciferase reporter plasmids and transfected into different liver-derived cell lines from human (HuH-7, HepG2 and Hep3B) and mouse origin (Hepal-6 and AML-12). As a reference the inventors used the reporter plasmid containing the cytomegalovirus promoter (CMV). The promoter-less plasmid pGL3-Basic was used to determine background luciferase activity. The inventors obtained different results depending on the cell line. Whereas the EAAT promoter was more potent than C27P in HepG2 and HuH-7 cells, no difference was observed in AML-12, and both promoters showed relatively low activity in Hep3B and Hepal-6 cells (FIG. 6B). In order to obtain more relevant information, the inventors performed in vivo transfection of plasmids in C57BL/6 mice by means of hydrodynamics injection. Quantification of light emission by BLI 48 h after injection revealed a strong transcriptional activity of the EAAT promoter and a relatively low strength of the C27P sequence (FIG. 6C), as expected based on the abundance of endogenous albumin and Cyp27a1 transcripts.

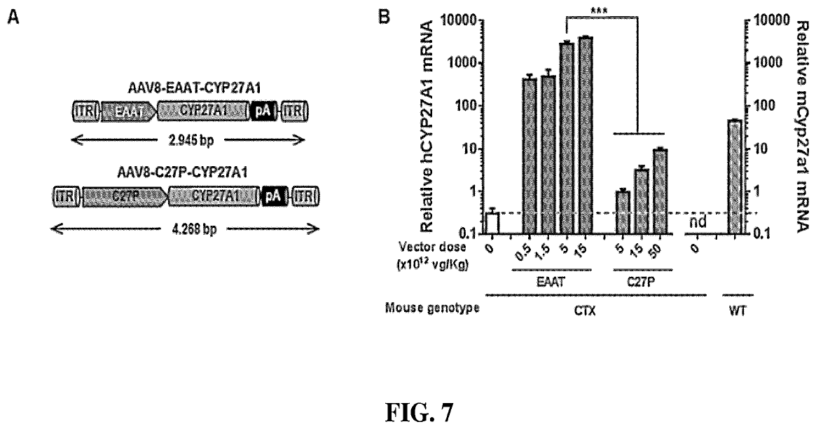

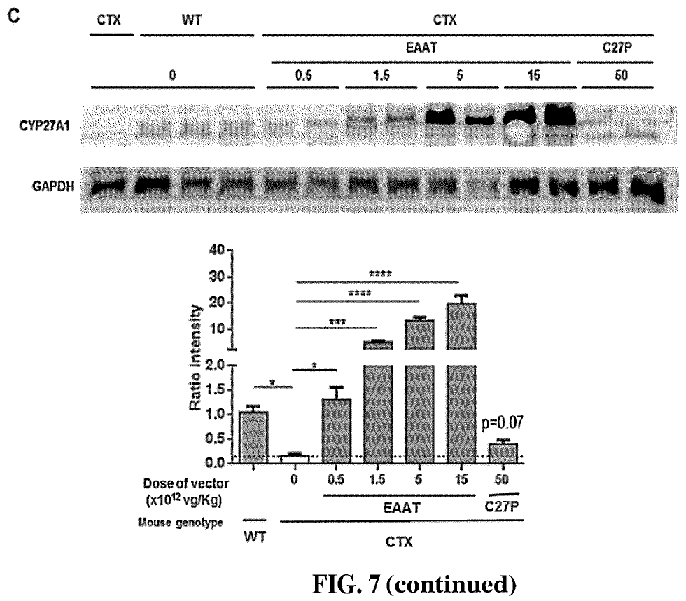

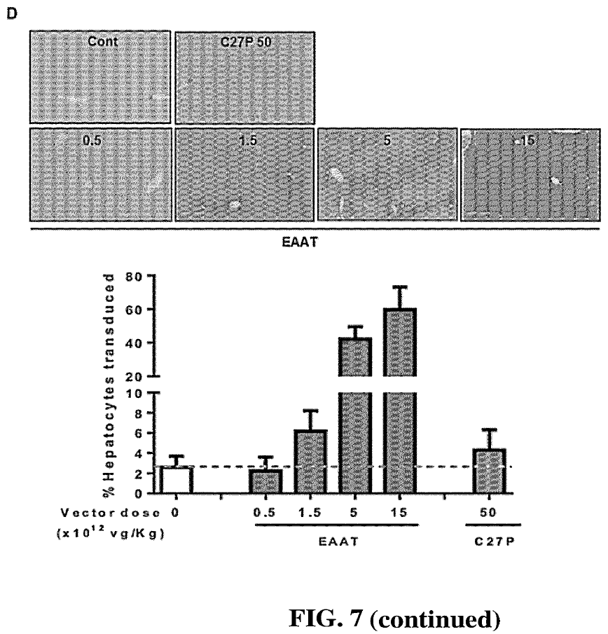

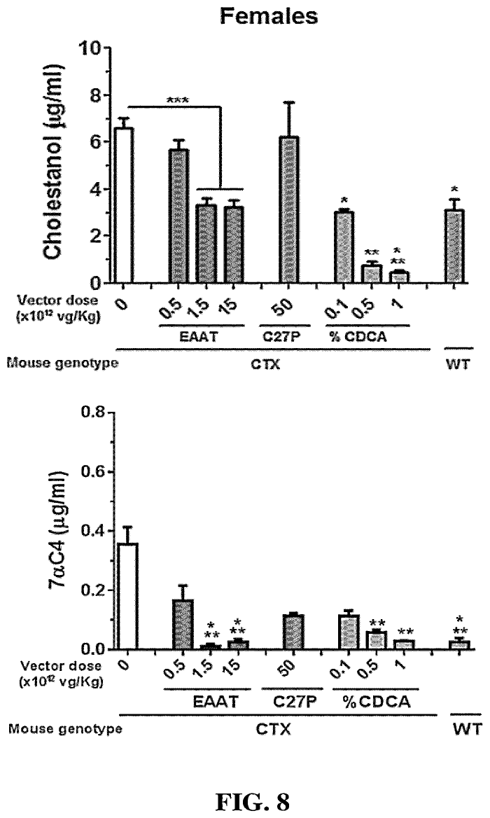

Both promoters were used to control the transcription of the CYP27A1 coding sequence, in the context of an AAV vector genome, giving rise to the AAV8-EAAT-CYP27A1 and AAV8-C27P-CYP27A1 vectors (FIG. 7A). Seven week-old CTX mice were treated with intravenous injections of the vectors at doses ranging from 5×10¹¹ to 5×10¹³ vg/Kg. Animals were sacrificed two weeks later, and transgene expression (CYP27A1) was analyzed by qRT-PCR in liver extracts.

As a reference for physiological expression, the endogenous mouse Cyp27a1 mRNA was quantified using primers targeted to exon 8 (Table 1). As expected, the full-length mouse Cyp27a1 mRNA was only detected in WT mice when these primers were used (FIG. 7B). Mice treated with the AAV8-C27P-CYP27A1 vector showed CYP27A1 mRNA levels above background only when the dose reached 5×10¹² vg/Kg. Even at the highest dose tested (5×10¹³ vg/Kg) the mRNA content was below the physiological level detected in WT mice. Although direct comparison of mouse and human mRNAs is not straightforward using qRT-PCR, these data indicate that the C27P regulatory sequence is weaker than the endogenous CYP27A1 promoter in its genomic context. In contrast, treatment with the AAV8-EAAT-CYP27A1 vector obtained a robust expression of CYP27A1 even at the lowest dose tested (5×10¹¹ vg/Kg, equivalent to 1×10¹⁰ vg per mouse). A fraction of liver samples was processed for protein extraction, and Western blot was performed in order to determine CYP27A1 content. Of note, the inventors used an antibody capable of detecting the mouse and orthologs, but not the truncated protein expressed by the CTX mice. The result partially confirms the strong expression in mice treated with the AAV8-EAAT-CYP27A1 vector. However, a global increase of CYP27A1 above WT levels was only observed when the dose of vector was 1.5×10¹² vg/Kg or higher (FIG. 7C). In agreement with the qRT-PCR results, the AAV8-C27P-CYP27A1 vector only achieved detectable CYP27A1 protein at the highest dose tested. In order to determine the percentage of transduced hepatocytes corresponding to these vector doses, liver samples were processed for immunohistochemistry. In mice treated with AAV8-EAAT-CYP27A1, hepatocytes over-expressing CYP27A1 could be readily detected, preferentially in the centrilobular zone (FIG. 7D). The inventors observed that a global increase of CYP27A1 could be obtained when less than 20% of hepatocytes over-express the transgene (1.5×10¹² vg/Kg vector dose). However, limitations in the sensitivity of the antibody precluded detection of hepatocytes expressing low levels, which resulted in a misleadingly low percentage of positive hepatocytes in mice treated with the AAV8-C27P-CYP27A1 vector. The inventors could not obtain consistent detection of mouse CYP27A1 protein in WT mice using the same antibody (not shown). To elucidate the suspected under-estimation of hepatocyte transduction, a new set of mice were treated with intravenous injections of the AAV8-EAAT-GFP vector, which expresses the reporter gene GFP under the control of the EAAT promoter. The high specificity and sensitivity of GFP immunohistochemical detection allowed confirmation that a dose of 1.5×10¹² vg/Kg vector transduces approximately 10% of mouse hepatocytes, whereas the 1.5×10¹³ vg/Kg dose reaches close to 80%.

The AAV8-EAAT-CYP27A1 Vector Normalizes Cholestanol and 7αC4 Levels in CTX Mice

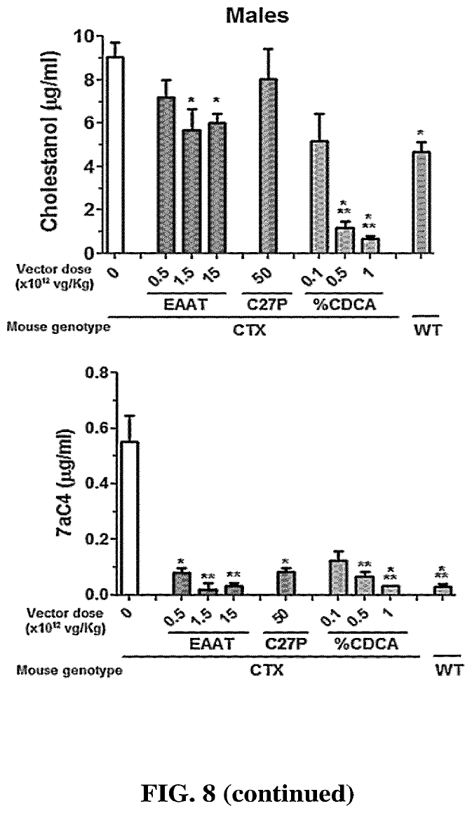

In order to assess the biological effect of the CYP27A1-expressing vectors, blood was collected from CTX mice two weeks after a single intravenous administration. Analysis of cholestanol and 7αC4 in plasma showed that AAV8-EAAT-CYP27A1 was able to normalize the metabolite levels at doses equal or higher than 1.5×10¹² vg/Kg in female and male mice (FIG. 8). The lowest dose of this vector (5×10¹¹ vg/Kg) achieved only a partial reduction, which was more evident in the case of 7αC4. The same trend was observed when the AAV8-C27P-CYP27A1 vector was used at the highest dose (5×10¹³ vg/Kg), in line with the relatively low expression of the transgene. The effects of gene therapy were compared with the standard treatment. To this aim, CTX mice were fed with diets enriched in CDCA at different percentages (0.1, 0.5 or 1% of chow weight) and followed for one month. The inventors observed a dose-dependent reduction of cholestanol and 7αC4 in plasma (FIG. 8). The decrease in cholestanol levels was especially intense, reaching values below WT mice at 0.5% CDCA or higher. In fact the inventors found that the therapeutic dose in the CTX model was 0.5%, since this dose was required to achieve full normalization of 7αC4 in mice (both male and female). Increasing the dose to 1% CDCA caused weight loss and was not further evaluated.

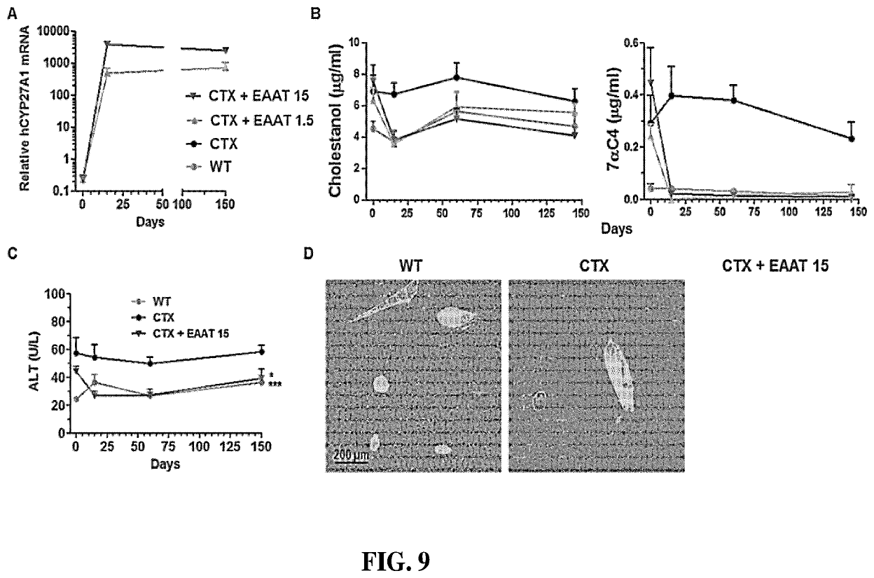

In order to determine the stability of transgene expression and therapeutic effect, additional groups of CTX mice treated with AAV8-EAAT-CYP27A1 at 1.5×10¹² or 1.5×10¹³ vg/Kg were sacrificed two weeks and 5 months after treatment. The analysis of liver and blood samples confirmed sustained transgene expression and correction of metabolites (FIGS. 9A and 9B, respectively). The AAV8-EAAT-CYP27A1 vector was well tolerated in CTX mice, with no elevation of serum transaminases (FIG. 9C), and absence of histopathological abnormalities in the liver (FIG. 9D). Untreated CTX mice showed higher ALT levels compared with WT littermates, but this mild elevation could be related to the presence of hepatomegaly, as discussed below.

The AAV8-EAAT-CYP27A1 Vector Restores Bile Acid Metabolism in CTX Mice

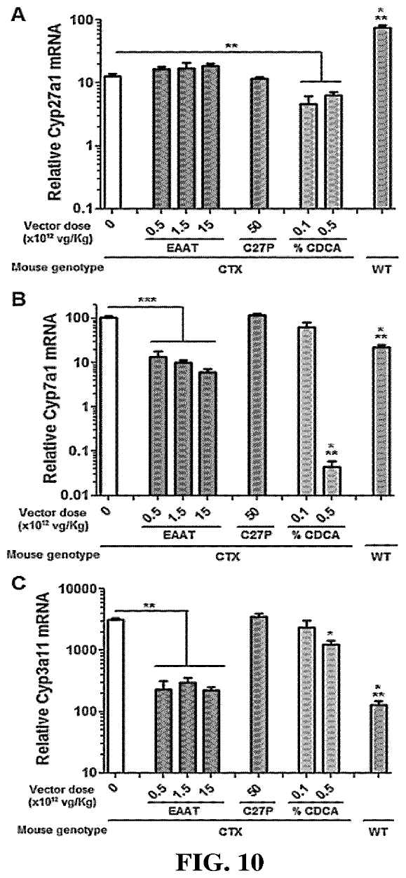

After confirming the biological effect of gene therapy and CDCA treatment on biochemical markers of CTX, the inventors studied the impact on the expression of key enzymes involved in bile acid metabolism. To this aim, mRNA was extracted from liver samples collected 2 weeks after vector administration, or one month after initiation of the CDCA treatment. First, the inventors studied the impact of the treatments on the transcriptional control of the endogenous Cyp27a1 gene. Since CTX mice present truncation of the gene at exon 8 out of 9 (Rosen H, et al. J. Biol. Chem. 1998; 273:14805-14812), the inventors employed primers targeting exons 1/2 in order to detect the wild type or truncated transcripts. In contrast with results shown in FIG. 7B (in which primers were designed in exons 8/9), Cyp27a1 mRNA could be detected in CTX mice. The transcripts were less abundant than in WT littermates, probably because of nonsense-mediated decay. The inventors observed that supplementation of CP27A1 had no influence on the transcription of the endogenous gene, whereas CDCA treatment caused a moderate inhibition (FIG. 10A). Quantification of Cyp7a1 expression confirmed up-regulation of this rate-limiting enzyme in CTX mice compared with their WT littermates (FIG. 10B). The AAV8-EAAT-CYP27A1 vector demonstrated efficient normalization of Cyp7a1 even at the lowest dose tested (5×10¹¹ vg/Kg), in agreement with the reduction of 7αC4 shown in FIG. 8. In contrast, a high dose of the AAV8-C27P-CYP27A1 vector was completely inefficient. Treatment with 0.5% CDCA caused a drastic reduction of Cyp7a1 expression, below physiological levels. Next, the inventors analyzed expression of the Cyp3a11 gene, which encodes a key enzyme in the response to xenobiotics in the liver (Sakamoto Y, et al. J. Toxicol. Sci. 2015; 40:787-796). As previously described (Honda A, et al. J. Biol. Chem. 2001; 276:34579-34585), CTX mice showed over-expression of this gene (FIG. 10C), thanks to the activation of the PXR pathway. Interestingly, 0.5% CDCA achieved only a slight reduction of Cyp3a11 expression, whereas AAV8-EAAT-CYP27A1 completely normalized mRNA content at all doses. These effects were entirely dependent on the efficient expression of CYP27A1 from AAV8-EAAT-CYP27A1, since an equivalent vector expressing GFP showed no changes compared with untreated CTX mice (data not shown). In addition, the AAV8-C27P-CYP27A1 vector showed no significant effect.

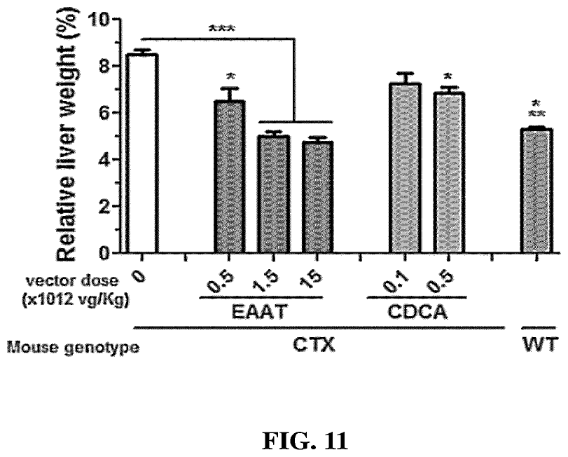

Activation of the xenobiotic response pathways produces hepatomegaly in CTX mice (Repa J J, et al. J. Biol. Chem. 2000; 275:39685-39692). This alteration was reversed by the AAV8-EAAT-CYP27A1 vector, but only partially by CDCA at 0.5% (FIG. 11).

The AAV8-EAAT-CYP27A1 Vector Normalizes Bile Acid Composition in Blood

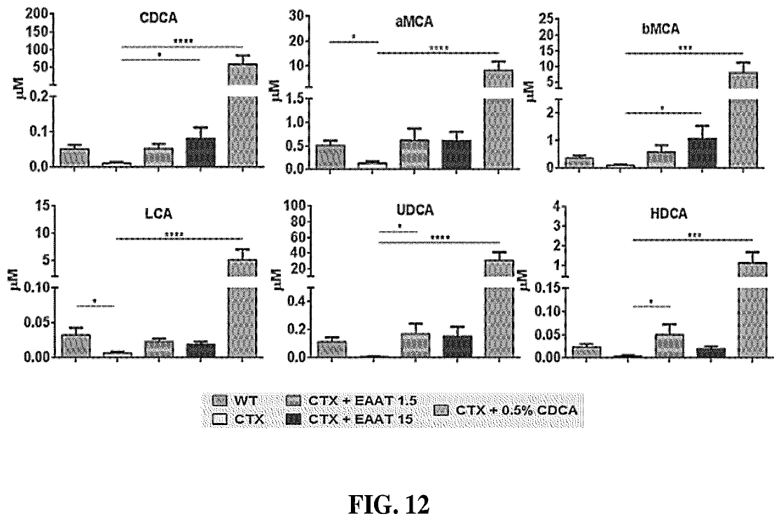

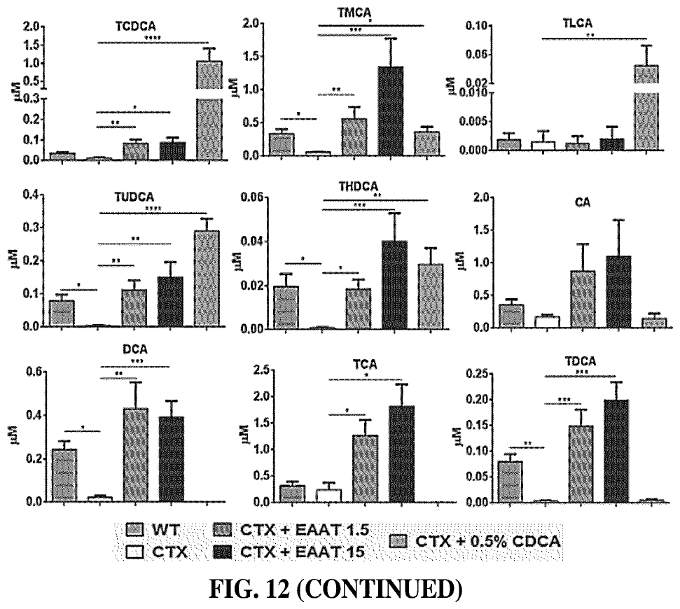

In order to determine if gene therapy is able to achieve sustained metabolic correction in CTX mice, the bile acid profile was analyzed in the blood of animals treated with AAV8-EAAT-CYP27A1 for 5 months at 1.5 or 15×10¹² vg/Kg. The optimal dose of CDCA (0.5% chow weight) was maintained for the same period and used for comparison. The inventors observed an increase of primary and secondary bile acids in mice treated with the vector at both doses (FIG. 12), including CDCA. In most cases the levels were equivalent to those found in WT littermates. Only the concentrations of cholic acid (CA), deoxycholic acid (DCA) and their tauroconjugates showed a moderate increase compared with WT mice at 1.5×10¹² vg/Kg. This tendency was more evident in the high dose group and included other species such as tauromuricholic acids (TMCA) and taurohyodeoxycholic acid (THDCA). In sharp contrast, treatment with CDCA caused a drastic increase in this bile acid and its derivatives (1000-fold above normal levels), whereas CA and derived species were not restored.

Discussion

The progress of the development of AAV vectors is making gene therapy a realistic option for monogenic diseases involving the liver. However, the clinical feasibility of this approach still requires careful preclinical evaluation. Apart from the size of the expression cassette (which should fit into the 4.7 Kb capacity of these vectors), one of the most important parameters is the percentage of transduced hepatocytes required to obtain a relevant therapeutic effect. The requirement of low percentages of hepatocyte transduction increase the chances of success using safe doses of the vectors. Typical examples are diseases in which a functional therapeutic protein can be expressed from the liver and secreted into the bloodstream, such as hemophilia (Peyvandi F, Garagiola I. Haemophilia. 2019; 25:738-746). In other cases such as the copper storage disorder Wilson's disease, the protein acts intracellularly, but transduced hepatocytes can act as a sink to eliminate the excess of copper (Murillo O, et al. J. Hepatol. 2016; 64; Murillo 0, et al. Hepatology. 2019; 70:108-126). The present results indicate that CTX could fall into the latter category, provided that the transduced hepatocytes express high enough amounts of the CYP27A1 cytochrome. The present preclinical results indicate that complete biochemical restoration can be obtained with less than 20% hepatocytes transduced by the AAV8-EAAT-CYP27A1 vector. This conclusion is based not only on CYP27A1 immunohistochemistry (which cannot detect hepatocytes expressing low levels), but also on indirect comparison with the AAV8-EAAT-GFP vector, which allows highly specific and sensitive GFP immunodetection. The inventors hypothesize that the excess of the highly permeable 7αC4 metabolite generated in untransduced hepatocytes can penetrate and be metabolized in other cells over-expressing CYP27A1. Still, this “sink effect” seems to have a limit, since the lowest dose of the AAV8-EAAT-CYP27A1 vector achieved a global hepatic content of CYP27A1 protein similar to the WT mice, but it obtained only a partial reduction in cholestanol levels. This indicates that the minimal threshold of transduced hepatocytes could be close to 10%, at least in the mouse model. In contrast, the effect of the AAV8-C27P-CYP27A1 vector was marginal even at the highest dose tested, probably because the transcriptional activation conferred by the C27P regulatory sequence was lower than the CYP27A1 promoter in its genomic context. Taking into account the size constraints imposed by the AAV cloning capacity, increasing the potency of this sequence would require the addition of enhancers, similar to the hybrid EAAT promoter (Kramer M G, et al. Mol. Ther. 2003; 7:375-385). Over-expression of CYP27A1 was well tolerated in CTX mice, suggesting that physiological regulation of transgene expression is not an absolute requisite in this disease. This is another advantageous circumstance in terms of clinical feasibility. The need for alternative therapies for CTX is apparently low because the standard treatment based on lifelong oral administration of CDCA is efficient in controlling cholestanol levels and ameliorates many clinical manifestations such as chronic diarrhea and progression of xanthomas (Verrips A, et al. Neurol. Sci. 2020; 41:943-949). However, in this work the inventors provide evidence that the mechanism of action of gene therapy is different. CDCA at the therapeutic dose caused a marked accumulation of this bile acid in blood, as observed in CTX patients (Batta A K, Tint G S. Metabolism. 1994; 43:1018-1022; Salen G, et al. J. Clin. Invest. 1974; 53:612-621). The inventors found that Cyp7a1 expression was virtually abrogated at the therapeutic dose. Despite this drastic effect, the xenobiotic response pathway remained activated in CTX mice, suggesting that the generation of other potentially toxic metabolites was not inhibited. This phenomenon could only be detected using a mouse model, since the PXR pathway is not induced in CTX patients (Honda A, et al. J. Biol. Chem. 2001; 276:34579-34585). Further investigation is needed to determine if these metabolites could be responsible for the progressive neurological deterioration observed in many patients despite CDCA treatment (Mignarri A, et al. J. Inherit. Metab. Dis. 2016; 39:75-83; Pilo-de-la-Fuente B, et al. Eur. J. Neurol. 2011; 18:1203-1211; Mignarri A, et al. J. Neurol. 2017; 264:862-874). Anecdotal experiences with plasmapheresis favor the hypothesis that complete detoxification is not achieved with CDCA treatment alone (Mimura Y, et al. J. Neurol. Sci. 1993; 114:227-230). According to the present preclinical results, dose escalation would not be an option because it is not well tolerated. Development of a mouse model with clear neurological manifestations is needed to assess if gene therapy is able to address this important aspect of the disease. Despite the existence of some differences between mouse and human bile acid metabolism, the inventors found that Cyp27a1^{-/-} mice are a valuable tool to evaluate different treatments at the biochemical level. For instance, 0.5% CDCA was very efficient in reducing cholestanol, but 7αC4 was not completely normalized, in line with some clinical observations (Mignarri A, et al. J. Inherit. Metab. Dis. 2016; 39:75-83). In contrast, gene therapy achieved a parallel reduction of both metabolites. In summary, the inventors provide evidence that CYP27A1 supplementation using an AAV vector could be a safe and feasible alternative for the treatment of CTX, offering the possibility of complete and stable metabolic correction after a single vector administration.

Sequences for Use in Practicing the Invention

Sequences for use in practicing the invention are described below:

Sterol 26-hydroxylase amino acid sequence (NCBI reference Sequence:
NP_000775.1 accessed on Apr. 25, 2020)
(SEQ ID NO: 1)
MAALGCARLRWALRGAGRGLCPHGARAKAAIPAALPSDKATGAPGAGPGVRRRQRSLEEIPR
LGQLRFFFQLFVQGYALQLHQLQVLYKAKYGPMWMSYLGPQMHVNLASAPLLEQVMRQEGKY
PVRNDMELWKEHRDQHDLTYGPFTTEGHHWYQLRQALNQRLLKPAEAALYTDAFNEVIDDFM
TRLDQLRAESASGNQVSDMAQLFYYFALEAICYILFEKRIGCLQRSIPEDTVTFVRSIGLMF
QNSLYATFLPKWTRPVLPFWKRYLDGWNAIFSFGKKLIDEKLEDMEAQLQAAGPDGIQVSGY
QVPQHKDFAHMPLLKAVLKETLRLYPVVPTNSRIIEKEIEVDGFLFPKNTQFVFCHYVVSRD
PTAFSEPESFQPHRWLRNSQPATPRIQHPFGSVPFGYGVRACLGRRIAELEMQLLLARLIQK
YKVVLAPETGELKSVARIVLVPNKKVGLQFLQRQC
Nucleotide sequence encoding CYP27A1
(SEQ ID NO: 2)
ATGGCTGCGCTGGGCTGCGCGAGGCTGAGGTGGGCGCTGCGAGGGGCCGGCCGTGGCCTCTG
CCCCCACGGGGCCAGAGCCAAGGCCGCGATCCCTGCCGCCCTCCCCTCGGACAAGGCCACCG
GAGCTCCCGGAGCCGGGCCTGGTGTCCGGCGGCGGCAACGGAGCTTAGAGGAGATTCCACGT
CTAGGACAGCTGCGCTTCTTCTTTCAGCTGTTCGTTCAAGGCTATGCCCTGCAACTGCACCA
GTTACAGGTGCTTTACAAGGCCAAGTACGGTCCAATGTGGATGTCCTACTTAGGGCCTCAGA
TGCACGTGAACCTGGCCAGTGCCCCGCTCTTGGAGCAAGTGATGCGGCAAGAGGGCAAGTAC
CCAGTACGGAACGACATGGAGCTATGGAAGGAGCACCGGGACCAGCACGACCTGACCTATGG
GCCGTTCACCACGGAAGGACACCACTGGTACCAGCTGCGCCAGGCTCTGAACCAGCGGTTGC
TGAAGCCAGCGGAAGCAGCGCTCTATACGGATGCTTTCAATGAGGTGATTGATGACTTTATG
ACTCGACTGGACCAGCTGCGGGCAGAGAGTGCTTCGGGGAACCAGGTGTCGGACATGGCTCA
ACTCTTCTACTACTTTGCCTTGGAAGCTATTTGCTACATCCTGTTCGAGAAACGCATTGGCT
GCCTGCAGCGATCCATCCCCGAGGACACCGTGACCTTCGTCAGATCCATCGGGTTAATGTTC
CAGAACTCACTCTATGCCACCTTCCTCCCCAAGTGGACTCGCCCCGTGCTGCCTTTCTGGAA
GCGATACCTGGATGGTTGGAATGCCATCTTTTCCTTTGGGAAGAAGCTGATTGATGAGAAGC
TCGAAGATATGGAGGCCCAACTGCAGGCAGCAGGGCCAGATGGCATCCAGGTGTCTGGCTAC
CTGCACTTCTTACTGGCCAGTGGACAGCTCAGTCCTCGGGAGGCCATGGGCAGCCTGCCTGA
GCTGCTCATGGCTGGAGTGGACACGACATCCAACACGCTGACATGGGCCCTGTACCACCTCT
CAAAGGACCCTGAGATCCAGGAGGCCTTGCACGAGGAAGTGGTGGGTGTGGTGCCAGCCGGG
CAAGTGCCCCAGCACAAGGACTTTGCCCACATGCCGTTGCTCAAAGCTGTGCTTAAGGAGAC
TCTGCGTCTCTACCCTGTGGTCCCCACAAACTCCCGGATCATAGAAAAGGAAATTGAAGTTG
ATGGCTTCCTCTTCCCCAAGAACACCCAGTTTGTGTTCTGCCACTATGTGGTGTCCCGGGAC
CCCACTGCCTTCTCTGAGCCTGAAAGCTTCCAGCCCCACCGCTGGCTGAGAAACAGCCAGCC
TGCTACCCCCAGGATCCAGCACCCATTTGGCTCTGTGCCCTTTGGCTATGGGGTCCGGGCCT
GCCTGGGCCGCAGGATTGCAGAGCTGGAGATGCAGCTACTCCTCGCAAGGCTGATCCAGAAG
TACAAGGTGGTCCTGGCCCCGGAGACGGGGGAGTTGAAGAGTGTGGCCCGCATTGTCCTGGT
TCCCAATAAGAAAGTGGGCCTGCA
GTTCCTGCAGAGACAGTGCTGA
CYP27A1 regulatory element (C27P)
(SEQ ID NO: 3)
TTAACTTTTGTGTCAAGGCATTTTTGAACAAGCAAGGGCTATCCCTAAAGTCATGAGGCAGT
ATTGTCCTGGTGTACCATTGGGTAATAGTAGTTTCAGGATTAGTCTACTTGAAAGGAAATAA
GTTCTGAGAGTCGAAGTGCAAAGGAATACAAAAGAAAGCATTCTTATGATCTAACACTGTGA
ACTAATTGGTGTTTTCTGGTATTTGGTTAAGTATAGTATAAGGATTTGGCACTATAGGATGT
AAGAGAATTATGACCTCGTTAATGGCTCCTAAGTCTTGAACTAGATCATATATATATATATA
TATATATAATTTTTTTTTTTTGAGATGGAGTCTCACTCTGTTGTCCAGGTTGGAGTGCATGG
CACAATCTCAGCTCACTGCAATCTCCGCCTCCCGGGTTCAAGCGATTCTCCTGCCTCAGTCT
CCCCAAGTAGCTGGGATCACGTGTGCCACCACGTTTTTAAAGCATGGCCTCCTGTCCTTCCT
GTCCAAAAACCAAATGAAAAATATGGTCTAGGTTGGGTGCTGTGGCTCACACCTGTAATTCC
AGCACTTTGGGAGGCCGAGGTAGGTAGATCATCTGAGGTCAGGAGTTTGAGACCAGCCTGGC
CAACATGGCAAAACCCTATCTCTACTAAAAATACAAAAATTAGCCGGGCGTGGTGGCGGGCA
CCTCTAATTCCAGCTACTCTGGAGGCTGAGACCGGAGAATTGCTTGAACCGGGAGGCGGAGG
TTGCAGCGAGCCAGGATTGCACCACTGCACTCCAGCCTGGACGACAGGGTGAGACTCTGGGG
AGTGGGGAGGAGAGAATTAACAAACTATATGACACAAAGGAACAAAAATACACAAGACAGGA
TAAGAGAGAGGAAGGATACAGGGAGAGCTAACATACATATAGCAGTTTGAAGTCCCGGGAAA
GGAGAAAAAAATGGGGAGGAGCAATATTTGAACAGATAATATCTGAGAAACTCCCCAGACTG
ATCAAAGACATCCAGTCACAGAGTCAAGAAACTAGGAGTCCTAGAATAAATAAGTAGAAAGC
CACACCTAGCAACTCAGCTGTCAGCCTAATTGAACTTAATATGCCCTTTCTTCTCTGGCTGC
CTGTAAATCACCAATTTTTCAAATGCTGTTAGAACCAGCTGTACCATCCTGCTGGGTCCTTC
ATTCCTGGCTTGAATGTGATCTTTGACCCTGTATTTATTTTATACTTGCGTGAGTTATGTTT
TTAAACTTTTTGACAACTATACTTTGAATATACATGAAACATAGGATTCATAAGACAATACT
TTTTTTTTCTTCTTGCTGTACGTTTTCCGTACTATGTTACTCTTTCCATTTTATTAAAAATA
ATTCTGGCCATCATCTACGAAATCTGGTCTCAACCTGCAGTTTGAAATATCCTGCCTTGGCA
GACGCGGCAGGGAGGTGCGTGGAGGGAGAATTGAATTAAAAAAAAAAACAACAAAACACGAA
TATTTAGATTTCTTTGTTCAGCGGCCCCCCTCCAGGGATCAGATGACTGGCCCCCCTCGCTC
CGAACTGACTCCGGGATCAATCCGGAAGGCCATTGGGAGAAGCCGAGGGCAGCTTAGCCACG
GCCGGTTCCCGTTCCCTCCAGGACGCGAGGGTCGCCTTGGGTGGGGAACCGCGACCGGGCGA
GGACCTATCCCGGTGTGGGGCTTCCCGATTTCGAAAGAATCTCGCTGCACCCCCGCCCAGAG
TTCAGACCAAGCGAAAAGTTATTTGAGAGGCCTCGGGGGCGCGGGGTGAGGAGTCGTGGCGG
AGGCCTTGGTCGGGGCGCCGTGGATATCCCCGAGTCACCGCGTCCCTCTCCTGCAGCTCCCG
CGTCGCTGGGAGGAGCGAGGGAGCGAGCGGGAAGGGGTCTAGCTGGCCTTTGCTCGGCCCTC
CCCAGCGCCCGGCTTTGAACCCGCCCTGCACTGCTGTCTGGGCGGGTCCGGGGACTCAGCAC
TCGACCCAAAGGTGCAGGCGCGCGAGCACAACCCGCTAGCGAATTC
Human alpha-1 anti-trypsin gene promoter
(SEQ ID NO: 4)
CGCCACCCCCTCCACCTTGGACACAGGACGCTGTGGTTTCTGAGCCAGGTACAATGACTCCT
TTCGGTAAGTGCAGTGGAAGCTGTACACTGCCCAGGCAAAGCGTCCGGGCAGCGTAGGCGGG
CGACTCAGATCCCAGCCAGTGGACTTAGCCCCTGTTTGCTCCTCCGATAACTGGGGTGACCT
TGGTTAATATTCACCAGCAGCCTCCCCCGTTGCCCCTCTGGATCCACTGCTTAAATACGGAC
GAGGACAGGGCCCTGTCTCCTCAGCTTCAGGCACCACCACTGACCTGGGACAGTGAA
Mouse serum albumin enhancer
(SEQ ID NO: 5)
GTTCCTAGATTACACTACACATTCTGCAAGCATAGCACAGAGCAATGTTCTACTTTAATTAC
TTTCATTTTCTTGTATCCTCACAGCCTAGAAAATAACCTGCGTTACAGCATCCACTCAGTAT
CCCTTGAGCATGAGGTGACACTACTTAACATAGGGACGAGATGGTACTTTGTGTCTCCTGCT
CTGTCAGCAGGGCACAGTACTTGCTGATACCAGGGAATGTTTGTTCTTAAATACCATCATTC
CGGACGTGTTTGCCTTGGCCAGTTTTCCATGTACATGCAGAAAGAAGTTTGGACTGATCAAT
ACAGTCCTCTGCCTTTAAAGCAATAGGAAAAGGCCAACTTGTCTACGTTTAGTATGTGGCTG
TAGATCTGTACC
Chimeric promoter sequence EATT
(SEQ ID NO: 6)
GTTCCTAGATTACACTACACATTCTGCAAGCATAGCACAGAGCAATGTTCTACTTTAATTAC
TTTCATTTTCTTGTATCCTCACAGCCTAGAAAATAACCTGCGTTACAGCATCCACTCAGTAT
CCCTTGAGCATGAGGTGACACTACTTAACATAGGGACGAGATGGTACTTTGTGTCTCCTGCT
CTGTCAGCAGGGCACAGTACTTGCTGATACCAGGGAATGTTTGTTCTTAAATACCATCATTC
CGGACGTGTTTGCCTTGGCCAGTTTTCCATGTACATGCAGAAAGAAGTTTGGACTGATCAAT
ACAGTCCTCTGCCTTTAAAGCAATAGGAAAAGGCCAACTTGTCTACGTTTAGTATGTGGCTG
TAGATCTGTACCCGCCACCCCCTCCACCTTGGACACAGGACGCTGTGGTTTCTGAGCCAGGT
ACAATGACTCCTTTCGGTAAGTGCAGTGGAAGCTGTACACTGCCCAGGCAAAGCGTCCGGGC
AGCGTAGGCGGGCGACTCAGATCCCAGCCAGTGGACTTAGCCCCTGTTTGCTCCTCCGATAA
CTGGGGTGACCTTGGTTAATATTCACCAGCAGCCTCCCCCGTTGCCCCTCTGGATCCACTGC
TTAAATACGGACGAGGACAGGGCCCTGTCTCCTCAGCTTCAGGCACCACCACTGACCTGGGA
CAGTGAA
5′-ITR
(SEQ ID NO: 7)
CCTGCAGGCAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTGGCCAACTC
CATCACTAGGGGTTCCTGCGGCCGC
3′-ITR
(SEQ ID NO: 8)
GCGGCCGCAGGAACCCCTAGTGATGGAGTTGGCCACTCCCTCTCTGCGCGCTCGCTCGCTCA
CTGAGGCCGGGCGACCAAAGGTCGCCCGACGCCCGGGCTTTGCCCGGGCGGCCTCAGTGAGC
GAGCGAGCGCGCAGCTGCCTGCAG
Nucleic acid construct: ITR-EAAT-CYP27A1-polyA-ITR
(SEQ ID NO: 9)
CCTGCAGGCAGCTGCGCGCTCGCTCGCTCACTGAGGCCGCCCGGGCAAAGCCCGGGCGTCGG
GCGACCTTTGGTCGCCCGGCCTCAGTGAGCGAGCGAGCGCGCAGAGAGGGAGTGGCCAACTC
CATCACTAGGGGTTCCTGCGGCCGCACGCGTGTTCCTAGATTACACTACACATTCTGCAAGC
ATAGCACAGAGCAATGTTCTACTTTAATTACTTTCATTTTCTTGTATCCTCACAGCCTAGAA
AATAACCTGCGTTACAGCATCCACTCAGTATCCCTTGAGCATGAGGTGACACTACTTAACAT
AGGGACGAGATGGTACTTTGTGTCTCCTGCTCTGTCAGCAGGGCACAGTACTTGCTGATACC
AGGGAATGTTTGTTCTTAAATACCATCATTCCGGACGTGTTTGCCTTGGCCAGTTTTCCATG
TACATGCAGAAAGAAGTTTGGACTGATCAATACAGTCCTCTGCCTTTAAAGCAATAGGAAAA
GGCCAACTTGTCTACGTTTAGTATGTGGCTGTAGATCTGTACCCGCCACCCCCTCCACCTTG
GACACAGGACGCTGTGGTTTCTGAGCCAGGTACAATGACTCCTTTCGGTAAGTGCAGTGGAA
GCTGTACACTGCCCAGGCAAAGCGTCCGGGCAGCGTAGGCGGGCGACTCAGATCCCAGCCAG
TGGACTTAGCCCCTGTTTGCTCCTCCGATAACTGGGGTGACCTTGGTTAATATTCACCAGCA
GCCTCCCCCGTTGCCCCTCTGGATCCACTGCTTAAATACGGACGAGGACAGGGCCCTGTCTC
CTCAGCTTCAGGCACCACCACTGACCTGGGACAGTGAAGCGGCCGCTAGCGAATTCTTGGCA
TTCCGGTACTGTTGGTAAAGCCACCATGGCTGCGCTGGGCTGCGCGAGGCTGAGGTGGGCGC
TGCGAGGGGCCGGCCGTGGCCTCTGCCCCCACGGGGCCAGAGCCAAGGCCGCGATCCCTGCC
GCCCTCCCCTCGGACAAGGCCACCGGAGCTCCCGGAGCCGGGCCTGGTGTCCGGCGGCGGCA
ACGGAGCTTAGAGGAGATTCCACGTCTAGGACAGCTGCGCTTCTTCTTTCAGCTGTTCGTTC
AAGGCTATGCCCTGCAACTGCACCAGTTACAGGTGCTTTACAAGGCCAAGTACGGTCCAATG
TGGATGTCCTACTTAGGGCCTCAGATGCACGTGAACCTGGCCAGTGCCCCGCTCTTGGAGCA
AGTGATGCGGCAAGAGGGCAAGTACCCAGTACGGAACGACATGGAGCTATGGAAGGAGCACC
GGGACCAGCACGACCTGACCTATGGGCCGTTCACCACGGAAGGACACCACTGGTACCAGCTG
CGCCAGGCTCTGAACCAGCGGTTGCTGAAGCCAGCGGAAGCAGCGCTCTATACGGATGCTTT
CAATGAGGTGATTGATGACTTTATGACTCGACTGGACCAGCTGCGGGCAGAGAGTGCTTCGG
GGAACCAGGTGTCGGACATGGCTCAACTCTTCTACTACTTTGCCTTGGAAGCTATTTGCTAC
ATCCTGTTCGAGAAACGCATTGGCTGCCTGCAGCGATCCATCCCCGAGGACACCGTGACCTT
CGTCAGATCCATCGGGTTAATGTTCCAGAACTCACTCTATGCCACCTTCCTCCCCAAGTGGA
CTCGCCCCGTGCTGCCTTTCTGGAAGCGATACCTGGATGGTTGGAATGCCATCTTTTCCTTT
GGGAAGAAGCTGATTGATGAGAAGCTCGAAGATATGGAGGCCCAACTGCAGGCAGCAGGGCC
AGATGGCATCCAGGTGTCTGGCTACCTGCACTTCTTACTGGCCAGTGGACAGCTCAGTCCTC
GGGAGGCCATGGGCAGCCTGCCTGAGCTGCTCATGGCTGGAGTGGACACGACATCCAACACG
CTGACATGGGCCCTGTACCACCTCTCAAAGGACCCTGAGATCCAGGAGGCCTTGCACGAGGA
AGTGGTGGGTGTGGTGCCAGCCGGGCAAGTGCCCCAGCACAAGGACTTTGCCCACATGCCGT
TGCTCAAAGCTGTGCTTAAGGAGACTCTGCGTCTCTACCCTGTGGTCCCCACAAACTCCCGG
ATCATAGAAAAGGAAATTGAAGTTGATGGCTTCCTCTTCCCCAAGAACACCCAGTTTGTGTT
CTGCCACTATGTGGTGTCCCGGGACCCCACTGCCTTCTCTGAGCCTGAAAGCTTCCAGCCCC
ACCGCTGGCTGAGAAACAGCCAGCCTGCTACCCCCAGGATCCAGCACCCATTTGGCTCTGTG
CCCTTTGGCTATGGGGTCCGGGCCTGCCTGGGCCGCAGGATTGCAGAGCTGGAGATGCAGCT
ACTCCTCGCAAGGCTGATCCAGAAGTACAAGGTGGTCCTGGCCCCGGAGACGGGGGAGTTGA
AGAGTGTGGCCCGCATTGTCCTGGTTCCCAATAAGAAAGTGGGCCTGCAGTTCCTGCAGAGA
CAGTGCTGAGCGGCCAacaatgcgatccgaTGGCCGCGACTCTAGAGTCGGGGCGGCCGGCC
GCTTCGAGCAGACATGATAAGATACATTGATGAGTTTGGACAAACCACAACTAGAATGCAGT
GAAAAAAATGCTTTATTTGTGAAATTTGTGATGCTATTGCTTTATTTGTAACCATTATAAGC
TGCAATAAACAAGTTAACAACAACAATTGCATTCATTTTATGTTTCAGGTTCAGGGGGAGGT
GTGGGAGGTTTTTTAAAGCAAGTAAAACCTCTACAAATGTGGTAAAATCGATAAGCCCGTGC
GGACCGAGCGGCCGCAGGAACCCCTAGTGATGGAGTTGGCCACTCCCTCTCTGCGCGCTCGC
TCGCTCACTGAGGCCGGGCGACCAAAGGTCGCCCGACGCCCGGGCTTTGCCCGGGCGGCCTC
AGTGAGCGAGCGAGCGCGCAGCTGCCTGCAG

1. A nucleic acid construct comprising a liver-specific promoter operably linked to a transgene encoding human sterol 27-hydroxylase or a functional variant thereof.

2. The nucleic acid construct of claim 1 wherein said liver-specific promoter comprises a human alpha-1-antitrypsin promoter.

3. The nucleic acid construct of claim 1 wherein said liver-specific promoter comprises a mouse albumin enhancer.

4. The nucleic acid construct of claim 1 wherein said liver-specific promoter comprises the mouse albumin enhancer and the human alpha-1 anti-trypsin promoter.

5. The nucleic acid construct of claim 1 wherein said liver-specific promoter comprises a nucleic acid sequence having at least 80% of identity with SEQ ID NO: 6.

6. The nucleic acid construct according to claim 1 further comprising a 5′ITR and a 3′ITR sequences from an adeno-associated virus.

7. The nucleic acid construct according to claim 1 comprising a nucleic acid sequence having at least 80% of identity with SEQ ID NO: 9.

8. An expression vector comprising a nucleic acid construct according to claim 1.

9. The expression vector of claim 8 wherein said vector is a viral vector.

10. A viral particle comprising the nucleic acid construct according to claim 1.

11. An AAV particle comprising the nucleic acid construct according to claim 1.

12. A host cell comprising the nucleic acid construct according to claim 1.

13. A pharmaceutical composition comprising the nucleic acid construct according to claim 1 and a pharmaceutically acceptable excipient.

14. A method for the prevention or the treatment of Cerebrotendinous Xanthomatosis (CTX) in a subject in need thereof comprising administering to the subject a therapeutically effective amount of the nucleic acid construct according to claim 1.

15. (canceled)

16. A method of producing viral particles, comprising the steps of:
a) culturing a packaging cell comprising the nucleic acid construct according to claim 1 in a culture medium, and
harvesting the viral particles from cell culture supernatant and/or inside the packaging cells;
c).

17. The nucleic acid according to claim 1 further comprising 5′ITR and a 3′ITR sequences from the AAV2 serotype.

18. The nucleic acid according to claim 1 further comprising 5′ITR and a 3′ITR sequences of SEQ ID NO: 7 and 8.

19. The expression vector of claim 8 wherein said vector is an adeno-associated viral (AAV) vector.

20. An AAV particle comprising the nucleic acid construct according to claim 1 and comprising capsid proteins of adeno-associated virus selected from the group consisting of: AAV3 type 3A, AAV3 type 3B, NP40, NP59, NP84, LK03, AAV3-ST, Anc80, AAV9 and AAV8 serotype.
